(19) United States
(12) Patent Application Publication
(10) Pub. No.: US 2026/0250764 A1
(43) Pub. Date: Aug. 27, 2026

(54) GENETIC SIGNATURES FOR PREDICTION OF DRUG RESPONSE OR RISK OF DISEASE

(71) Applicants: National University of Singapore, Singapore (SG); Tan Tock Seng Hospital Pte. Ltd., Singapore (SG); Agency for Science, Technology and Research, Singapore (SG)

(72) Inventors: Guat Lay Caroline LEE, Singapore (SG); Jun Wei Ashley LIM, Singapore (SG); Lee Jin LIM, Singapore (SG); Siong Chuan Samuel CHONG, Singapore (SG); Khai Pang LEONG, Singapore (SG); Chiea Chuen KHOR, Singapore (SG)

(73) Assignees: National University of Singapore, Singapore (SG); Tan Tock Seng Hospital Pte. Ltd., Singapore (SG); Agency for Science, Technology and Research, Singapore (SG)

(21) Appl. No.: 18/727,319

(22) PCT Filed: Jan. 6, 2023
(86) PCT No.: PCT/SG2023/050015

§ 371 Date: Jul. 8, 2024

(30) Foreign Application Priority Data
Jan. 6, 2022 (SG) 10202200113S

(51) Int. Cl. C12Q 1/6883; G16B 20/20; G16H 10/60

(52) U.S. Cl. CPC C12Q 1/6883; G16B 20/20; G16H 10/60; C12Q 2600/106; C12Q 2600/118; C12Q 2600/156

(57) ABSTRACT

The present invention relates generally to genomics. In particular, the specification teaches a method of predicting subjects at risk of rheumatoid arthritis and the responsiveness of a subject towards methotrexate (MTX) treatment.

### FIELD OF INVENTION

The present invention relates generally to genomics. In particular, the specification teaches a method of predicting subjects at risk of rheumatoid arthritis and the responsiveness of a subject towards methotrexate (MTX) treatment.

### BACKGROUND

Rheumatoid arthritis (RA) is one of the most common chronic inflammatory joint diseases, affecting ~1% of the population worldwide with mortality approximately 1.5 times that of the general population. RA is a complex autoimmune disease primarily characterised by the swelling of the joints leading to joint pain, stiffness and in severe cases irreversible joint damage. This is further exacerbated by several associated comorbidities such as coronary artery diseases and hyperlipidaemia. The onset of RA is typically in middle age, thus it disproportionally affects adults at the peak of their productivity, and the effects of the disease can pose a major socioeconomic burden on both the patient and on society.

Early identification of at-risk individuals and timely provision of preventive or mitigation measures is critical to minimise the effects of RA. Methotrexate (MTX), a folate analogue with immunomodulatory and anti-inflammatory properties, was introduced to the management of RA more than 40 years ago, and is still recommended for the initial treatment of moderate—to high-severity RA. Nonetheless, approximately one-third of patients fail to respond to MTX treatment. There is no single diagnostic test for RA and clinicians rely on patterns of clinical presentation for diagnosis. Physicians also cannot identify poor or non-responders to MTX treatment at the outset, so there is frequently a delay in the initiation of alternative therapies.

It would be desirable to overcome or ameliorate at least one of the above-described problems, or at least to provide a useful alternative.

### SUMMARY

Disclosed herein is a method of predicting the responsiveness of a subject towards methotrexate (MTX) treatment, the method comprising a) detecting the allele and the number of alleles at a plurality of SNP loci listed in Table 1 or Table 2, using a sample obtained from the subject, and b) generating a risk score for the subject based on the allele and the number of alleles at the plurality of SNP loci, wherein the risk score, as compared to a reference, provides an indication as to whether the subject is responsive or non-responsive towards MTX treatment.

Disclosed herein is a method of treating a subject in need of therapy, the method comprising a) detecting the allele and the number of alleles at a plurality of SNP loci listed in Table 1 or Table 2, using a sample obtained from the subject; b) generating a risk score for the subject based on the allele and the number of alleles at the plurality of SNP loci, wherein the risk score, as compared to a reference, provides an indication as to whether the subject is responsive or non-responsive towards MTX treatment; and c) treating the subject when the subject is predicted to be responsive or non-responsive towards MTX.

Disclosed herein is a method of detecting a subject at risk of rheumatoid arthritis, the method comprising a) detecting the allele and the number of alleles at a plurality of SNP loci listed in Table 3, using a sample obtained from the subject, and b) generating a risk score for the subject based on the allele and the number of alleles at the plurality of SNP loci, wherein the risk score, as compared to a reference, provides an indication as to whether the subject is at risk of rheumatoid arthritis.

Disclosed herein is a method of treating a subject at risk of rheumatoid arthritis, the method comprising a) detecting the allele and the number of alleles at a plurality of SNP loci listed in Table 3, using a sample obtained from the subject; b) generating a risk score for the subject based on the allele and the number of alleles at the plurality of SNP loci, wherein the risk score, as compared to a reference, provides an indication as to whether the subject is at risk of rheumatoid arthritis; and c) treating the subject found to be at risk of rheumatoid arthritis.

Disclosed herein is a computer-implemented method for determining a genetic signature for predicting the responsiveness of a subject towards methotrexate (MTX) treatment, the method comprising: a) receiving exome sequencing records of a plurality of subjects responsive towards MTX treatment (case records) and a plurality of subjects not responsive to MTX treatment (control records), wherein the case records and control records are identified with case record and control record labels; b) merging the case records and control records to obtain a training dataset; c) aligning records in the training dataset to the human reference genome to obtain an aligned training dataset; d) identifying potentially functional SNPs (pfSNPs) and/or coding haplotypes in each of the records in the aligned training dataset, wherein each identified pfSNP and/or coding haplotype is treated as a candidate feature; e) performing feature selection among the pfSNPs and/or coding haplotypes using a recursive feature selection algorithm and the case and control record labels to select a plurality of predictive pfSNPs and/or coding haplotypes predictive of responsiveness towards MTX treatment; f) defining a genetic signature based on the selected plurality of predictive pfSNPs and/or coding haplotypes predictive of responsiveness towards MTX treatment.

Disclosed herein is a computer-implemented method for determining a genetic signature for predicting a subject at risk of rheumatoid arthritis, the method comprising: a) receiving exome sequencing records of a plurality of subjects suffering from rheumatoid arthritis (case records) and a plurality of subjects not suffering from rheumatoid arthritis (control records), wherein the case records and control records are identified with case record and control record labels; b) merging the case records and control records to obtain a training dataset; c) aligning records in the training dataset to the human reference genome to obtain an aligned training dataset; d) identifying SNPs in each of the records in the aligned training dataset, wherein each identified SNP is treated as a candidate feature; e) performing feature selection among the SNPs using a recursive feature selection algorithm and the case and control record labels to select a plurality of predictive SNPs predictive of rheumatoid arthritis; f) defining a genetic signature based on the selected plurality of predictive SNPs predictive of rheumatoid arthritis.

### BRIEF DESCRIPTION OF THE DRAWINGS

Embodiments of the present invention will now be described, by way of non-limiting example, with reference to the drawings in which:

FIG. 1 shows a workflow to identify the best pfSNP predictors of methotrexate response. Patient samples were first divided into training and test sets, followed by generation of eight training subsets with variable sample sizes. The important features were selected from pfSNPs or pfSNPs with non-genetic factors using recursive feature elimination with cross-validation (RFECV). The predictive performance of important features that were commonly identified in all subsets was assessed in six different machine learning models. Finally, the minimal subset of features, which are pfSNPs integrated with non-genetic factors were tested in unseen test set.

FIG. 2 shows the predictive performance of 56 pfSNPs with 5 non-genetic training factors in a training set. ROC curves for 56 pfSNPs with 5 non-genetic training factors were obtained using (A) Support Vector Machine, (B) Neural Network, (C) Boosted Trees, (D) Random Forest, (E) Logistic Regression, and (F) Elastic Net. Black box: Model that achieves AUC≥0.9.

FIG. 3 shows the predictive performance of 56 pfSNPs with 5 non-genetic training factors in a test set. ROC curves for 56 pfSNPs with 5 non-genetic training factors were obtained using (A) Support Vector Machine, (B) Neural Network, (C) Boosted Trees, (D) Random Forest, (E) Logistic Regression, and (F) Elastic Net. Black box: Model that achieves AUC≥0.9.

FIG. 4 shows pathways that involve the genes associated with the 56 pfSNPs.

FIG. 5 shows the predictive performance of 95 haplotypes and 5 non-genetic factors in a training set using 5-fold cross-validation. ROC curves of 95 haplotypes and 5 non-genetic factors were obtained using (A) Random Forest, (B) Logistic Regression, (C) Support Vector Machine, (D) Boosted Trees, (E) Elastic Net, and (F) Neural Network.

FIG. 6 shows the predictive performance of 95 haplotypes and 5 non-genetic factors in an unseen test set. ROC curves of 95 haplotypes and 5 non-genetic factors were obtained using (A) Random Forest, (B) Logistic Regression, (C) Support Vector Machine, (D) Boosted Trees, (E) Elastic Net, and (F) Neural Network.

FIG. 7 shows pathways that involve the genes of the 95 haplotypes.

FIG. 8 shows ROC-AUC, Sensitivity, and Specificity scores using an increasing number of the commonly selected SNPs from RFECV based on their mean feature importance scores for prediction of RA. Each of the commonly selected SNPs from RFECV were gradually included based their feature importance scores (from highest to lowest) in the evaluation of using 5-fold cross-validation of training set, unseen test set 1, unseen test set 2, and unseen test set 3. Evaluation scores (ROC-AUC, Sensitivity, Specificity) were plotted against the number of selected SNPs (#SNPs) included in the prediction model. Dotted vertical line in each plot represented the determined optimal number of SNPs for a good evaluation score.

FIG. 9 shows the redictive performance of 9 selected SNPs in the training set using 5-fold cross-validation.

ROC-AUC curves with Accuracy, Sensitivity, and Specificity of 9 selected SNPs using (a) Logistic Regression, (b) Naïve Bayes, (c) Random Forest, (d) XGBoost, and (e) Support Vector Machine (SVM) classifiers.

FIG. 10 is a summary of the impact of the 9 selected SNP features on Random Forest model output. (a) Average impact on both model output (population control and RA cases); (b) Impact on Population control classification based on feature values; (c) Impact on RA cases classification based on feature values.

FIG. 11 shows the distribution of AUC scores obtained from 1,000 sets of randomly selected 9 SNPs. Distribution of AUC-ROC scores binned in intervals of 0.01 of 1,000 sets of randomly selected 9 SNPs to verify that predictive performance observed from the selected 9 SNPs by the feature selection pipeline employed was a non-random occurrence. AUC-ROC scores were obtained by evaluation in each of the three unseen test sets using one of the five chosen ML classifiers, the Random Forest classifier.

FIG. 12 shows distribution plots of PRS scores binned in intervals of 0.1 for RA case samples and population control samples

FIG. 13 shows the predictive performance of using PRS in the training set using 5-fold cross-validation. ROC-AUC curves with Accuracy, Sensitivity, and Specificity of PRS using (a) Logistic Regression, (b) Naïve Bayes, (c) Random Forest, (d) XGBoost, and (e) Support Vector Machine (SVM) classifiers.

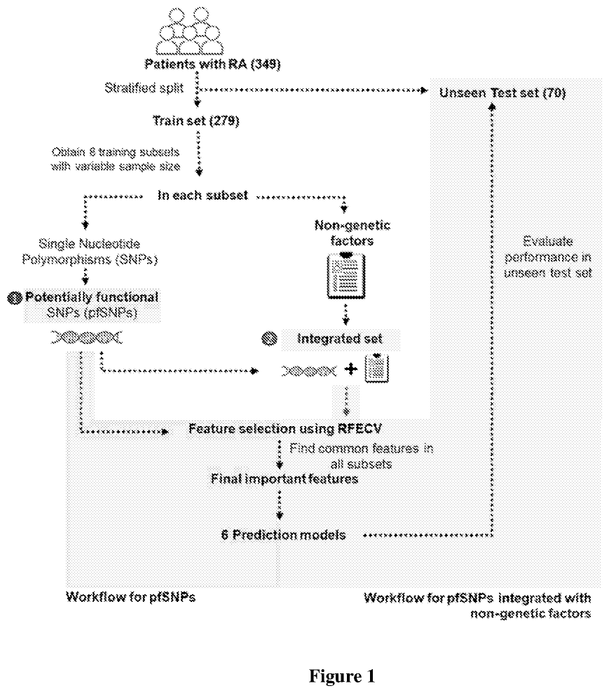

### DETAILED DESCRIPTION

Disclosed herein is a method of predicting the responsiveness of a subject towards methotrexate (MTX) treatment, the method comprising a) detecting the allele and the number of alleles at a plurality of SNP loci listed in Table 1 or Table 2, using a sample obtained from the subject, and b) generating a risk score for the subject based on the allele and the number of alleles at the plurality of SNP loci, wherein the risk score, as compared to a reference, provides an indication as to whether the subject is responsive or non-responsive towards MTX treatment.

As used herein, the term “autoimmune disease” refers to diseases and disorders induced by the body's immune responses being directed against its own tissues, causing prolonged inflammation and subsequent tissue destruction. Non limiting examples of autoimmune diseases and disorders include alopecia areata, diabetes Type 1, Guillain-Barre syndrome, multiple sclerosis, rheumatoid arthritis and systemic lupus erythematosus among others. In one embodiment, the subject is suffering from rheumatoid arthritis (RA). In another embodiment the subject is an RA patient indicated for or undergoing MTX treatment.

The term “gene” as used herein refers to a region of a DNA sequence that encodes a polypeptide or protein, intronic sequences, promoter regions, and upstream (i.e., proximal) and downstream (i.e., distal) non-coding transcription control regions (e.g., enhancer and/or repressor regions). A “protein-coding sequence” or “coding sequence” refers to that part of a gene that encodes a polypeptide or protein, including regions transcribed as introns, exons and terminal untranslated regions in mRNA. A “non-coding sequence” refers to a DNA or RNA sequence which does not encode a polypeptide or protein. This includes sequences known to be non-coding (e.g. regulatory regions, sequences encoding non-coding RNA, scaffold attachment regions, satellite or microsatellite regions, telomeric regions, fragments of transposons and retrotransposons) and also sequences with unknown function.

The term “genetic variation” or “nucleotide variation” refers to a change in a nucleotide sequence (e.g., an insertion, deletion, inversion, duplication, or substitution of one or more nucleotides) relative to a reference sequence, which may be, e.g., a commonly-found and/or wild-type sequence, and/or the sequence of a major allele. The term also encompasses the corresponding change in the complement of the nucleotide sequence, unless otherwise indicated.

A “genetic marker” is an identifiable DNA sequence which is genetically variable (i.e., polymorphic) for different individuals within a population. A marker at the DNA sequence level is linked to a specific chromosomal location unique to an individual's genotype and inherited in a predictable manner. Genetic markers include, for example, single nucleotide polymorphisms (SNPs), indels (i.e., insertions/deletions), simple sequence repeats (SSRs), restriction fragment length polymorphisms (RFLPs), random amplified polymorphic DNAs (RAPDs), amplified fragment length polymorphisms (AFLPs), variable number tandem repeats (VNTRs), single-strand conformation polymorphisms (SSCPs), among many other examples. In general, to be useful, a genetic marker needs to have two or more alleles or variants.

The term “allele” refers to a sequence variant of a DNA sequence. Alleles can but need not be located within a gene sequence. Alleles can be identified with respect to a single polymorphic position, genetic marker or marker sequence. The term “polymorphism” refers to the presence in a population of two or more allelic variants.

As used herein a “single nucleotide polymorphism”, or “SNP”, refers to a single base position in a DNA molecule at which alternative nucleotides exist in a population. A “SNP locus” refers to a chromosomal region where a SNP is located. An allele of a SNP refers to the actual nucleotide that is present, i.e. a SNP allele may be A, T, C, or G. The methods herein involve detecting the alleles and the number of alleles at a plurality of SNP loci. A subject may be homozygous or heterozygous for an allele at each SNP locus, wherein “homozygous” refers to the presence of two identical SNP alleles at a SNP locus, and “heterozygous” refers to the presence of two different SNP alleles at a SNP locus.

SNPs may fall within coding sequences of genes, non-coding regions of genes, or in the intergenic regions between genes. SNPs within a coding sequence will not necessarily change the amino acid sequence of the protein that is produced, due to degeneracy of the genetic code. SNPs in the coding region are of two types, synonymous and non-synonymous SNPs. Synonymous SNPs do not affect the protein sequence while non-synonymous SNPs change the amino acid sequence of protein. Non-synonymous SNPs are also called coding SNPs. SNPs that are not in protein-coding regions may still affect gene splicing, transcription factor binding, mRNA degradation, or the sequence of non-coding RNA. An “expression-associated SNP” or “expression SNP” or “eSNP” is a SNP that affects gene expression. An eSNP may be in a coding or non-coding region.

Reference may be made to either DNA strand when referring to a particular SNP position, SNP allele, or nucleotide sequence. A SNP may be identified by its “RSID”, consisting of the prefix “rs” followed by a unique identification number. The RSID is a non-redundant and globally unique accession number assigned to SNPs registered at the U.S. National Center for Biotechnology Information (NCBI), and accessible at the NCBI's SNP database (https://www.nebi.nlm.nih.gov/snp/). An RSID identifies all the alleles of a SNP that map to the same location on the genome. For example, the rs10221770 SNP refers to both the “C” and “G” alleles at that SNP locus (instead of an “A” allele). For the purposes of the methods herein, where an RSID identifies more than one SNP allele, the detection of any of those alleles may be used to identify the presence or absence of a SNP and/or used for predictive evaluation. Reference to an RSID herein includes all the SNP alleles identified by that RSID, as well as all current and future RSIDs that may be used to identify the underlying SNP alleles.

As used herein a “potentially functional SNP” or “pfSNP” is a SNP which has the capacity to induce a change in the expression, structure, function or activity of a gene, gene isoform, protein, or protein isoform. Regions that may host pfSNPs include but are not limited to protein-coding regions, regions coding for sites of post-translational modification, regions coding for functional protein domains, regions that affect nonsense-mediated RNA decay, promoter regions, gene regulatory regions, transcription factor binding sites, microRNA binding sites, regions involved in transcript processing, regions involved in RNA processing, exonic splice enhancer/silencer (ESE/ESS) sites, and regions responsible for RNA turnover, transport or sequestration. A pfSNP database may be derived from published databases using computational approaches. Such approaches are described in Wang, J. et al. (Hum Mutat. 2011 January, 32(1):19-24 (2011)) and Bachtiar, M. et al. (Pharmacogenomics J. 19(6): 516-527 (2019)), which are incorporated by reference herein. Databases which may be used to identify pfSNPs are described in Yue, P. et al. (BMC Bioinformatics, 7:166 (2006)), Oscanoa, J. et al. (Nucleic Acids Res, 48(W1):W185-W92 (2020)), Võsa, U. et al. (BioRxiv 44736 (2018)), Lonsdale, J. et al. (Nat Genet, 45:580-5 (2013)), Carithers, L. J. et al. (Biopreserv Biobank, 13 (5): 311-9 (2015)) and Jansen, R. et al. (Hum Mol Genet, 26(8): 1444-51 (2017)).

The functionality of a pfSNP may be evaluated using methods known in the art. Such methods may be in vitro and in vivo methods performed on DNA, RNA, a protein, cell, tissue, organ or organism. Non-limiting examples of such methods include biochemical assays (e.g., immunoprecipitation, electrophoretic mobility shift or super-shift assays, ligand binding assays), gene expression assays, protein functional assays, and bioinformatic studies (e.g., genomic, transcriptomic, or proteomic studies). The methods may involve the use of model cellular systems, model tissue systems or model organisms.

As used herein, a “haplotype” refers to a SNP or a combination of SNPs at various loci on the same chromosome that are inherited as a unit. A haplotype may be one locus, several loci, or an entire chromosome depending on the number of recombination events that have occurred between a given set of loci, if any occurred. A “coding haplotype” herein is a haplotype consisting of one or more SNPs in protein-coding regions. The SNPs in a coding haplotype may be located in a single gene or in different genes, and may be synonymous or non-synonymous SNPs. For a specific gene segment, there are often many theoretically possible combinations of SNPs, and therefore there are many theoretically possible haplotypes.

A “predictive SNP” or “predictive pfSNP” or “predictive coding haplotype” refers to a SNP or pfSNP or coding haplotype identified among the SNPs or pfSNPs or coding haplotypes that is potentially predictive of responsiveness of a subject towards methotrexate (MTX) treatment or the risk of a subject developing rheumatoid arthritis. The predictive SNP, pfSNP or coding haplotype may possess biological meaning relevant for prediction of responsiveness to MTX or risk of rheumatoid arthritis. The predictive SNPs, pfSNPs or coding haplotypes are identified by the application of recursive feature selection on SNPs, pfSNPs or coding haplotypes present in a training dataset of case records and control records.

A recursive feature selection algorithm is an algorithm to select features by recursively considering smaller and smaller sets of features with reference to an estimator. An estimator is trained on the initial set of features and the importance of each feature is obtained based on their predictive significance. Then, the least important features are pruned from a current set of features. That procedure is recursively repeated on the pruned set until the desired number of features to select is eventually reached. In some embodiments, a random forest classifier may be incorporated as an estimator.

A SNP may be associated with a phenotype or phenotypic trait. The term “phenotype” refers to any visible, detectable or otherwise measurable property of an organism. A “phenotypic trait” or “trait” refers to a distinct phenotypic variant. The term “genotype” refers to the genetic constitution of an organism. This may be considered in total (i.e., at a genome level), or with respect to the alleles of a single gene (i.e., at a given genetic locus). The term “associated with” in connection with a relationship between a SNP and a phenotype or trait refers to a statistically significant dependence of SNP frequency on a quantitative scale or qualitative gradation of the phenotype. A SNP “positively” correlates with a trait when it is linked to it and when the presence of the SNP is an indicator that the desired trait or trait form will occur in an organism comprising the SNP. A SNP “negatively” correlates with a trait when it is linked to it and when presence of the SNP is an indicator that a desired trait or trait form will not occur in an organism comprising the SNP.

As used herein, the phrase “quantitative trait” refers to a phenotypic trait that can be measured or described numerically. A “quantitative trait locus” or “QTL” refers to a locus that correlates with variation of a quantitative trait in a population of organisms. An “expression quantitative trait locus” or “eQTL” refers to a locus that correlates with variation of gene expression of one or more genes.

A unique set of SNPs and/or haplotypes may be used as a genetic signature indicative of a phenotypic trait. As used herein a “genetic signature” or “SNP signature” denotes a collection of one or more SNPs and/or haplotypes, the combination of which is associated with a phenotype or trait. A trait may have more than one genetic signature. Reference to a SNP with respect to a genetic signature also includes the genotype and alleles of that SNP. Reference to a haplotype with respect to a genetic signature also includes all the SNPs and the SNP alleles that form that haplotype. A genetic signature may be used to identify a trait in a cell, tissue or organism, when the trait is latent or hidden or yet to manifest.

The present methods provide for SNPs, haploptypes and genetic signatures that are associated with rheumatoid arthritis (RA) and treatment outcomes following methotrexate (MTX) treatment. There is provided a method for determining a subject's propensity to respond to MTX treatment, said method comprising a screen for the alleles and number of alleles at a plurality of SNP loci set forth in either Table 1 or Table 2. Also provided herein is a method for identifying a subject at risk of developing RA, said method comprising a screen for the alleles and number of alleles at at a plurality of SNP loci set forth in Table 3 and Table 4. For the avoidance of doubt, the detection of an allele at a SNP locus set forth in Table 1, 2, 3 or 4 includes the detection of all SNP alleles identified by the RSID of that SNP in Table 1, 2, 3 or 4.

In some embodiments the method may include inspecting a data set indicative of genetic characteristics previously derived from analysis of the subject's genome. A data set of genetic characteristics of the subject may include, for example, a listing of SNPs in the subject's genome or a complete or partial sequence of the subject's genomic DNA. In other embodiments the method may include obtaining or isolating nucleic acid (e.g., DNA or RNA) from a sample from the subject and analysing it to determine whether the nucleic acid contains informative SNPs.

As used herein, an “individual”, “subject” or “patient” is a vertebrate. In certain embodiments, the vertebrate is a mammal. Mammals include, but are not limited to, primates (including human and non-human primates) and rodents (e.g., mice and rats). In some embodiments, a subject is a human.

A “sample” herein refers to a biological sample, typically derived from a biological fluid, cell, tissue, organ, or organism, containing a nucleic acid or a mixture of nucleic acids containing the SNPs that are to be detected in the method herein. Non-limiting examples of biological samples include blood, blood fractions, sputum, tissue biopsy, tissue explant, mucosal swab, mucosal smear, connective tissue, muscle, nervous tissue, epithelia, bone, cartilage, gastrointestinal tissue, reproductive tissue, vascular tissue, or any other tissue or cell preparation, or fraction or derivative thereof or isolated therefrom. The sample may be used directly as obtained from the subject or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilisation, concentration, amplification, nucleic acid fragmentation, inactivation of interfering components, the addition of reagents, lysis, etc. If such methods of pretreatment are employed with respect to the sample, such pretreatment methods are typically such that the nucleic acid(s) of interest remain in the test sample, sometimes at a concentration proportional to that in an untreated test sample (e.g., namely, a sample that is not subjected to any such pretreatment). A sample expressly encompasses fractions or processed portions thereof. In one embodiment the sample is a blood sample.

The method herein may involve isolation of nucleic acids from a sample for SNP genotyping. Nucleic acid can be obtained or isolated from a sample using any method of nucleic acid isolation known to one skilled in the art. Examples of nucleic acid isolation methods can be found in general laboratory manuals, such as Sambrook and Russel, Molecular Cloning: A Laboratory Manual, 3rd Ed., Cold Spring Harbor Laboratory Press, Cold Spring Harbor, N.Y. (2001). The step of isolating nucleic acids is not restricted to any particular degree of purity of the isolated nucleic acids.

Genomic DNA generally is used in the analysis of nucleotide sequence variants, although mRNA also can be used. Genomic DNA is typically extracted from a biological sample such as a peripheral blood sample, but can be extracted from other biological samples, including tissues (e.g., mucosal scrapings of the lining of the mouth or from prostate tissue). Routine methods can be used to extract genomic DNA from a blood or tissue sample, including, for example, phenol extraction. Alternatively, genomic DNA can be extracted with kits such as the QIAAMP® Tissue Kit (Qiagen, Chatsworth, Calif.) and the WIZARD® Genomic DNA purification kit (Promega).

An amplification step is typically, but not necessarily, performed before proceeding with the detection method. For example, exons or introns of a gene can be amplified and then directly sequenced. Dye primer sequencing can be used to increase the accuracy of detecting heterozygous samples.

PCR conditions and primers can be developed that amplify a product only when the variant allele is present or only when the wild type allele is present (e.g. allele-specific PCR). For example, patient DNA and a control can be amplified separately using either a wild type primer or a primer specific for the variant allele. Each set of reactions is then examined for the presence of amplification products using standard methods to visualize the DNA. For example, the reactions can be electrophoresed through an agarose gel and the DNA visualized by staining with ethidium bromide or other DNA intercalating dye. In DNA samples from heterozygous patients, reaction products would be detected in each reaction. Patient samples containing solely the wild type allele would have amplification products only in the reaction using the wild type primer. Similarly, patient samples containing solely the variant allele would have amplification products only in the reaction using the variant primer. Allele-specific PCR also can be performed using allele-specific primers that introduce priming sites for two universal energy-transfer-labeled primers (e.g., one primer labeled with a green dye such as fluorescein and one primer labeled with a red dye such as sulforhodamine). Amplification products can be analyzed for green and red fluorescence in a plate reader.

Mismatch cleavage methods also can be used to detect differing sequences by PCR amplification, followed by hybridization with the wild type sequence and cleavage at points of mismatch. Chemical reagents, such as carbodiimide or hydroxylamine and osmium tetroxide can be used to modify mismatched nucleotides to facilitate cleavage.

The methods described herein can be carried out using a computer programmed to receive data (e.g., data from a chip containing a panel of SNPs, indicating whether a subject contains SNP alleles in this disclosure). The computer can output for display information related to a subject's SNP signature and associated risk score.

The data may be obtained via any technique that results in an individual receiving data associated with a sample. For example, an individual may obtain the dataset by generating the dataset himself by methods known to those in the art. Alternatively, the dataset may be obtained by receiving a dataset or one or more data values from another individual or entity. For example, a laboratory professional may generate certain data values while another individual, such as a medical professional, may input all or part of the dataset into an analytic process to generate the result.

The term “SNP genotyping” or “SNP detection” refers to the identification and/or quantitation of a SNP allele in a sample. A suitable detection method is able to determine whether a SNP allele identified by an RSID in Table 1, 2, 3 or 4 is present or absent in the sample, and also the number of copies of each SNP in a sample (i.e., if the subject is homozygous or heterozygous for that SNP).

The SNP allele may be directly detected and/or quantitated, or may be copied and/or amplified to allow detection of amplified copies of the allele. Detection can be performed by any means known to one skilled in the art, including, for example, restriction fragment length polymorphism (RFLP) analysis using restriction enzymes, single-strand conformational polymorphism (SSCP) analysis, heteroduplex analysis, chemical cleavage analysis, microarray analysis using hybridisation probes, oligonucleotide ligation and hybridisation assays, allele-specific amplification, mass spectrometry methods, or next-generation sequencing technologies applied to a whole genome or exome. Combinations of these methods may also be used.

A number of suitable high throughput formats exist for genotyping SNPs. Generally, such methods involve a logical or physical array of oligonucleotides (e.g., primers or probes), the subject samples, or both. Common array formats include both liquid and solid phase arrays. For example, assays employing liquid phase arrays, e.g., for hybridization of nucleic acids, can be performed in multiwell or microtiter plates. Microtiter plates with 96, 384 or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis. Exemplary systems include, e.g., xMAP® technology from Luminex (Austin, Tex.), the SECTOR® Imager with MULTI-ARRAY® and MULTI-SPOT® technologies from Meso Scale Discovery (Gaithersburg, Md.), the ORCA™ system from Beckman-Coulter, Inc. (Fullerton, Calif.) and the ZYMATE™ systems from Zymark Corporation (Hopkinton, Mass.).

A variety of solid phase arrays can favorably be employed to genotype SNPs in the context of the disclosed methods. Exemplary formats include membrane or filter arrays (e.g., nitrocellulose, nylon), pin arrays, and bead arrays (e.g., in a liquid “slurry”). Typically, oligonucleotide probes corresponding to gene product are immobilized, for example by direct or indirect cross-linking, to the solid support. Essentially any solid support capable of withstanding the reagents and conditions necessary for performing the particular expression assay can be utilized. For example, functionalized glass, silicon, silicon dioxide, modified silicon, any of a variety of polymers, such as (poly)tetrafluoroethylene, (poly) vinylidenedifluoride, polystyrene, polycarbonate, or combinations thereof can all serve as the substrate for a solid phase array.

In some instances data may not be available for the allele or number of alleles present at one or more target SNP loci, e.g., SNP genotyping may fail to detect an allele at a target SNP locus, or there may be an error in genotyping such that data for a SNP locus has to be disregarded. In such instances, the allele may be regarded as “missing” or “undetected” for that target SNP locus, and the calculation of the risk score may be adjusted to take into account such missing or undetected alleles. Collister, J. A. et al. (Front Genet, February 18; 13:818574 (2022)) provides examples of risk score calculations for missing genotypes.

“Patient response” can be assessed using any endpoint indicating a benefit to the patient, including, without limitation, (1) inhibition, to some extent, of disease progression, including slowing down and complete arrest; (2) reduction in the number of disease episodes and/or symptoms; (3) relief, to some extent, of one or more symptoms associated with the disorder; (4) improvement, to some extent, in the status of one or more disease biomarkers; (5) increase in the length of disease-free presentation following treatment; and/or (6) decreased mortality at a given point of time following treatment. The assessment of patient response may include a physical assessment of the patient (e.g., a clinical evaluation) and/or an analysis of a sample from the patient (e.g., a biochemical analysis of a blood sample, or a histological analysis of a tissue sample).

The method as defined herein may predict a subject as one who is likely to be responsive or non-responsive to MTX treatment. Likelihood is suitably based on mathematical modeling. An increased likelihood, for example, may be relative or absolute and may be expressed qualitatively or quantitatively.

A “risk score” herein is used to define a subject's risk of developing rheumatoid arthritis or a subject's tendency to respond to methotrexate treatment. Both genetic and non-heritable factors that contribute to and/or are associated with disease risk or treatment outcome may be used to calculate a risk score. Genetic factors include but are not limited to point genetic variations in a subject's genome (e.g., SNPs).

In some embodiments, the risk score is a polygenic risk score. A “polygenic risk score” (PRS) herein refers to a risk score based on a number of genetic variants, the combination of which has significant predictive value for disease risk or treatment response. Methods of calculating polygenic risk scores are described in Choi, S. W. et al., Nat Protoc, September; 15(9):2759-2772 (2020). In some embodiments, the polygenic risk score is calculated by weighting the number of alleles present at each relevant SNP locus by the effect size of the allele, and is adjusted according to the number of SNP loci used to generate the risk score. For example, the PRS may be calculated using the following equation:

$$PRS = \frac{{\sum }_{i}^{j} {S}_{i} \times {G}_{ik}}{P \times {M}_{k}}$$

wherein Sᵢ is the effect size of SNP allele i in a sample k; j is the number of SNP loci used; Gik is number of copies of SNP allele i; P is the ploidy of a subject (which is two for a human subject); and Mₖ is the number of loci with detectable alleles in the sample. The effect size (or weight) of each allele may be obtained from genome-wide association studies (GWAS) in large discovery samples, wherein the effect size is determined based on statistical analysis of the strength of association of an allele with a phenotype of interest.

The calculated risk score may be compared to a reference (e.g., a reference score from a control cohort that does not have rheumatoid arthritis or is non-responsive to methotrexate treatment) to determine risk of disease or responsiveness to treatment. Alternatively, the risk score may be compared to a pre-determined value. In some embodiments a computer algorithm (e.g. a machine learning algorithm) is used to determine treatment response or disease risk based on the risk score, e.g. by generating a probability value for risk of disease or responsiveness to treatment after comparing the risk score to a reference.

A higher risk score or lower risk score as compared to a reference may indicate that a subject is likely to be responsive to treatment to MTX.

A higher risk score or lower risk score as compared to a reference may indicate that a subject is likely to be non-responsive to treatment to MTX.

In one embodiment, a higher risk score as compared to a reference indicates that a subject is at risk of a disease (e.g. rheumatoid arthritis).

In one embodiment, the method comprises genotyping or detecting SNPs at a locus selected from the group consisting of rs10221770, rs2286362, rs1056538, rs35618062, rs11618506, rs560096, rs7872034, rs3752293, rs12305038, rs9358856, rs73118352, rs2487302, rs12347076, rs325400, rs1557567, rs2240735, rs6116, rs11146986, rs13293151, rs929387, rs8192646, rs10412915, rs2232103, rs2236527, rs34968651, rs3796099, rs7722287, rs28446896, rs2853445, rs4681297, rs4796601, rs8191489, rs881863, rs9371061, rs2777729, rs474242, rs4850901, rs11808959, rs2072713, rs2271508, rs2286342, rs2286874, rs28507496, rs3730353, rs4821704, rs77273876, rs8039777, rs11983326, rs3802482, rs15881, rs62145930, rs879620, rs4412304, rs2168518, rs1291362 and rs165704. In some embodiments the method comprises detecting SNPs at all 56 SNP loci listed in Table 1.

In one embodiment, the method comprises genotyping or detecting SNPs at a locus selected from the group consisting of rs4617548, rs3731986, rs2279348, rs7935, rs9332464, rs10929378, rs2014572, rs3746231, rs10421632, rs8100154, rs10045774, rs4811, rs687, rs3829738, rs17099370, rs2032360, rs56204700, rs3801296, rs10268268, rs6115, rs6112, rs6116, rs5931046, rs2269415, rs10455097, rs2917862, rs2694657, rs2232108, rs2232105, rs2232103, rs2291289, rs17855750, rs619483, rs643232, rs1050457, rs2238567, rs3737002, rs3737002, rs941909, rs1054124, rs1133170, rs4669, rs4934, rs1059369, rs1804826, rs3751884, rs289318, rs2572023, rs216250, rs526690, rs557881, rs10277, rs1650893, rs10060182, rs3745403, rs2474328, rs1042780, rs10909567, rs7252988, rs343376, rs12918876, rs3812953, rs1134074, rs7380824, rs78233829, rs11554776, rs1043836, rs3739287, rs2974617, rs7716270, rs9462088, rs3729740, rs4906422, rs3742947, rs11621644, rs314452, rs8102923, rs8103177, rs2277921, rs12608777, rs61740752, rs12609001, rs2232762, rs1866844, rs2890827, rs957448, rs2304764, rs4085749, rs1061770, rs17857295, rs2326369, rs6779903, rs16843645, rs12943620, rs1131609, rs2292954, rs12960, rs8105737, rs41291993, rs1132358, rs231591, rs584961, rs1133763, rs2229276, rs12070777, rs5756130, rs2269529, rs2269530, rs602128, rs679620, rs63750222, rs61744212, rs2306857, rs1979572, rs2277923, rs10867826, rs2304155, rs2304154, rs4804151, rs4804152, rs17847215, rs3008815, rs10821135, rs730106, rs5498, rs639225, rs13058434, rs10753668, rs9449444, rs9361904, rs2273165, rs231228, rs35297478, rs7852399, rs8565, rs3820285, rs2272251, rs3746906, rs231235, rs1126605, rs3765114 and rs8042919. In some embodiments the method comprises genotyping or detecting SNPs at all 142 SNP loci listed in Table 2.

In some embodiments the method comprises genotyping or detecting all 95 coding haplotypes listed in Table 2.

The method may comprise detecting 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142 or more SNPs or haplotypes.

As used herein a “non-genetic factor” refers to a non-heritable variable that may be associated with a phenotype or trait. Non-genetic factors may include a range of demographic and clinical variables specific to a patient, non-limiting examples of which include patient gender, age, marital status, number of live births, menopause status, smoking history, family history of disease, levels of specific biomarkers, clinical presentation and treatment history with particular drugs or drug combinations. Non-genetic factors may be detected or quantitated, for example, through consultation with a subject, through a review of a subject's clinical or medication history, or through clinical testing of a sample from the subject

In some embodiments the methods herein further comprise the use of five non-genetic factors to generate a risk score for the subject. Non-genetic factors include but are not limited to: age, gender, marital status, menopause status or smoking history of a subject; number of children by the subject; family history of rheumatoid arthritis, lupus or osteoporosis; duration of morning stiffness experienced by a subject; functional status of an RA subject as set out by the American College of Rheumatology; treatment history of a subject with prednisolone, methotrexate, sulphasalzine, hydroxychloroquine, leflunomide, intramuscular gold, penicillamine, azathioprine, cyclophosphamide, infliximab, etanercept, COX2 inhibitors or non-steroidal anti-inflammatory drugs; platelet count of a subject; and levels of haemoglobin, rheumatoid factor or anti-cyclic citrullinated peptides in a subject.

In one embodiment, the method comprises the detection of SNPs at a plurality of SNP loci in Table 1 as well as the use of the following five non-genetic factors in the generation of a risk score: age of subject, duration of morning stiffness experienced by subject, number of children by subject, platelet count of subject, and haemoglobin levels of subject. In another embodiment, the method comprises the detection of SNPs at a plurality of SNP loci in Table 2 as well as the use of the following five non-genetic factors in the generation of a risk score: age of subject, duration of morning stiffness experienced by subject, levels of anti-cyclic citrullinated peptides in subject, platelet count of subject, and haemoglobin levels of subject.

Disclosed herein is a method of treating a subject in need of therapy, the method comprising a) detecting the allele and the number of alleles at a plurality of SNP loci listed in Table 1 or Table 2, using a sample obtained from the subject; b) generating a risk score for the subject based on the allele and the number of alleles at the plurality of SNP loci, wherein the risk score, as compared to a reference, provides an indication as to whether the subject is responsive or non-responsive towards MTX treatment; and c) treating the subject when the subject is predicted to be responsive or non-responsive towards MTX.

The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

Appropriate treatment methods may be any method that is known in the art for producing a desired therapeutic response without undue adverse side effects (such as toxicity, irritation or allergic response). Such treatment may be a monotherapy of MTX, a composition comprising MTX and one or more additional pharmaceutical agents, a combination therapy of MTX with one or more additional pharmaceutical agents, or non-MTX pharmacologic regimens. Appropriate treatments may further comprise non-pharmacologic and/or rehabilitative interventions, non-limiting examples of which include physical and occupational therapy, nutritional and dietary counselling, psychosocial interventions, and the use of orthoses and assistive devices. Examples of appropriate treatment methods for a subject predicted to be responsive to MTX are described in Visser, K. et al. (Ann Rheum Dis, 68(7):1094-9, July 2009) and Cipriani, P. et al. (Clin Ther; 36(3):427-35, March 2014). Examples of appropriate treatment methods for a subject predicted to be non-responsive to MTX are described in Aletaha, D. et al. (J Am Med Assoc 320:1360-72 (2018)).

Disclosed herein is a method of detecting a subject at risk of rheumatoid arthritis, the method comprising a) detecting the allele and the number of alleles at a plurality of SNP loci listed in Table 3, using a sample obtained from the subject, and b) generating a risk score for the subject based on the allele and the number of alleles at the plurality of SNP loci, wherein the risk score, as compared to a reference, provides an indication as to whether the subject is at risk of rheumatoid arthritis.

In some embodiments, the method further comprises detecting, in a sample obtained from the subject, the allele and the number of alleles at a plurality of SNP loci listed in Table 4 to generate a risk score for the subject.

A subject may be at risk of rheumatoid arthritis if there is at least a 5% chance of the subject developing rheumatoid arthritis over the subject's lifetime. A subject at risk of rheumatoid arthritis may have about a 5% chance, a 10% chance, a 15% chance, a 20% chance, a 25% chance, a 30% chance, a 35% chance, a 40% chance, a 45% chance, a 50% chance, a 55% chance, a 60% chance, a 65% chance, a 70% chance, a 75% chance, a 80% chance, a 85% chance, a 90% chance, a 95% chance, or a 100% chance of developing rheumatoid arthritis over the subject's lifetime.

In one embodiment, the method comprises genotyping or detecting SNPs at a locus selected from the group consisting of rs1266832853, rs200901373, rs60465633, rs142869031, rs11385557, rs143773270, rs11246240, rs112667995, rs79555231. In some embodiments the method comprises genotyping or detecting SNPs at all 9 SNP loci listed in Table 3. In some embodiments the method further comprises detecting at least one additional SNP selected from the group consisting of rs879004458, rs68004247, rs151105471 and 15:82890763:C:A.

In one embodiment, the method comprises genotyping or detecting SNPs at a locus selected from the group consisting of rs1266832853, rs200901373, rs60465633, rs142869031, rs11385557, rs143773270, rs11246240, rs112667995, rs79555231, rs879004458, rs68004247, rs151105471 and 15:82890763:C:A. In some embodiments the method comprises genotyping or detecting SNPs at all 13 SNP loci listed in Tables 3 and 4.

Disclosed herein is a method of treating a subject at risk of rheumatoid arthritis, the method comprising a) detecting the allele and the number of alleles at a plurality of SNP loci listed in Table 3, using a sample obtained from the subject; b) generating a risk score for the subject based on the allele and the number of alleles at the plurality of SNP loci, wherein the risk score, as compared to a reference, provides an indication as to whether the subject is at risk of rheumatoid arthritis; and c) treating the subject found to be at risk of rheumatoid arthritis Examples of RA treatment methods are described in Aletaha, D. et al. (J Am Med Assoc 320:1360-72 (2018)).

Also disclosed herein is a computer-implemented method for determining a genetic signature for predicting the responsiveness of a subject towards methotrexate (MTX) treatment, the method comprising:
a) receiving exome sequencing records of a plurality of subjects responsive towards MTX treatment (case records) and a plurality of subjects not responsive to MTX treatment (control records), wherein the case records and control records are identified with case record and control record labels;
b) merging the case records and control records to obtain a training dataset;
c) aligning records in the training dataset to the human reference genome to obtain an aligned training dataset;
d) identifying potentially functional SNPs (pfSNPs) and/or coding haplotypes in each of the records in the aligned training dataset, wherein each identified pfSNP and/or coding haplotype is treated as a candidate feature;
e) performing feature selection among the pfSNPs and/or coding haplotypes using a recursive feature selection algorithm and the case and control record labels to select a plurality of predictive pfSNPs and/or coding haplotypes predictive of responsiveness towards MTX treatment;
f) defining a genetic signature based on the selected plurality of predictive pfSNPs and/or coding haplotypes predictive of responsiveness towards MTX treatment.

Provided herein is the use of a genetic signature as defined herein for predicting the responsiveness of a subject towards methotrexate (MTX) treatment. The use may comprise detecting the allele and the number of alleles at a plurality of SNP loci in the genetic signature in a sample obtained from the subject, and generating a risk score for the subject based on the allele and the number of alleles at the plurality of SNP loci, wherein the risk score, as compared to a reference, provides an indication as to whether the subject is responsive or non-responsive towards MTX treatment.

Disclosed herein is also a computer-implemented method for determining a genetic signature for predicting a subject at risk of rheumatoid arthritis, the method comprising:
a) receiving exome sequencing records of a plurality of subjects suffering from rheumatoid arthritis (case records) and a plurality of subjects not suffering from rheumatoid arthritis (control records), wherein the case records and control records are identified with case record and control record labels;
b) merging the case records and control records to obtain a training dataset;
c) aligning records in the training dataset to the human reference genome to obtain an aligned training dataset;
d) identifying SNPs in each of the records in the aligned training dataset, wherein each identified SNP is treated as a candidate feature;
e) performing feature selection among the SNPs using a recursive feature selection algorithm and the case and control record labels to select a plurality of predictive SNPs predictive of rheumatoid arthritis;
f) defining a genetic signature based on the selected plurality of predictive SNPs predictive of rheumatoid arthritis.

Provided herein is the use of a genetic signature as defined herein for predicting a subject at risk of rheumatoid arthritis. The use may comprise detecting the allele and the number of alleles at a plurality of SNP loci in the genetic signature in a sample obtained from the subject, and generating a risk score for the subject based on the allele and the number of alleles at the plurality of SNP loci, wherein the risk score, as compared to a reference, provides an indication as to whether the subject is at risk of rheumatoid arthritis.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described. It is to be understood that the invention includes all such variations and modifications, which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

Certain embodiments of the invention will now be described with reference to the following examples which are intended for the purpose of illustration only and are not intended to limit the scope of the generality hereinbefore described.

### EXAMPLES

### Example 1

### Methodology

Study cohort

349 RA patients receiving MTX treatment were selected for SNP analysis. All patients received at least 3 months of MTX treatment at 15 mg per week and over 90% of the patients completed 2 years of treatment. MTX drug response is defined as remission within or at two years post-treatment, which is determined by evaluation of Disease Activity Score in 28 joints (DAS28). DAS28 is a composite score representative of RA activity that includes the number of tender joints and swollen joints, erythrocyte sedimentation rate, and a global assessment of health. Responders to MTX were classified as patients with DAS28<2.6 following MTX treatment. The following non-genetic features patients were also recorded: gender, age, marital status, number of children, menopause status, smoking status, social assistance status, functional status as defined by the American College of Rheumatology, family history of RA, family history of lupus, family history of osteoporosis, duration of morning stiffness, levels of anti-cyclic citrullinated peptides (anti-CCP), levels of rheumatoid factor, blood haemoglobin value, platelet count, prednisole treatment status, MTX treatment status, sulfasalazine treatment status, hydroxychloroquine treatment status, leflunomide treatment status, intra-muscular gold treatment status, penicillamine treatment status, imuran treatment status, CycA treatment status, Endoxan treatment status, Remicade treatment status, etanercept treatment status, NSAID treatment status, and COX2 inhibitor treatment status.

### Exome Sequencing

The exome region of genomic DNA from peripheral blood mononuclear cells was enriched using the Nimblegen SeqCap EZ kit (Roche) and with Agilent SureSelect Human All Exon kit (Agilent Technologies, CA). Products were then purified using AMPure XP system (Beckman Coulter, Beverly, USA) and quantified using the Agilent high sensitivity DNA assay on the Agilent Bioanalyzer 2100 system. Exome sequencing was performed by commercial providers using the IlluminaHiSeq2000 100PE platform.

pfSNPs

Using the exome sequencing data of RA patients, single nucleotide variants were identified, then pfSNPs were identified from an improved database developed in the laboratory.

### Haplotypes

Using single nucleotide variants called from the exome sequencing data, genotype phasing was performed using the BEAGLE 5.1 software. Phased genotypes from whole exome sequencing were utilised to establish haplotypes comprising single nucleotide polymorphisms (SNPs) within coding genes. Using PLINK v1.07 software, the variants within coding regions of each gene were each defined as an individual haplotype block. Only haplotypes with a minor haplotype frequency >0.01 were further analyzed. Haplotypes were further filtered for non-reference haplotypes (i.e., haplotypes that are not composed entirely of reference alleles).

### Data Processing

The total dataset from 349 subjects was split into a training set (80%, N=279) and an unseen test set (20%, N=70) in a stratified manner to maintain the ratio between MTX responders and non-responders. To avoid data leakage, subsequent processing steps were performed on the training and test datasets separately. The training dataset (80%) was further processed into 8 subsets of variable sample sizes through stratified random sampling with replacement. Within each training subset, features with near zero variance (i.e., features which are almost constant across all samples) or display >95% correlation with other features were excluded. To identify a signature of robust features that are important for prediction, we utilised recursive feature elimination with cross-validation (RFECV), removing 500 low importance features at each recursion iteration, incorporating a Random Forest classifier as the estimator. The process was performed using a 5-fold cross-validation until an optimal number of features was selected. To obtain features with high stability of importance, features that are common across all 8 training subsets were selected for further evaluation of their predictive performance.

### Evaluation

Six different machine learning classifiers were selected for predicting drug response. Given that there is currently no consensus as to which machine learning algorithm is best used with genomic data, the following six models were selected with the intention of providing a broad representation of popular machine learning algorithms used in prediction. These models included regression-based methods (Logistic Regression and Elastic Net), ensemble-based methods (Boosted Trees and Random Forest), and other popular machine learning algorithms (Neural Networks and Support Vector Machine).

### Results

pfSNPs

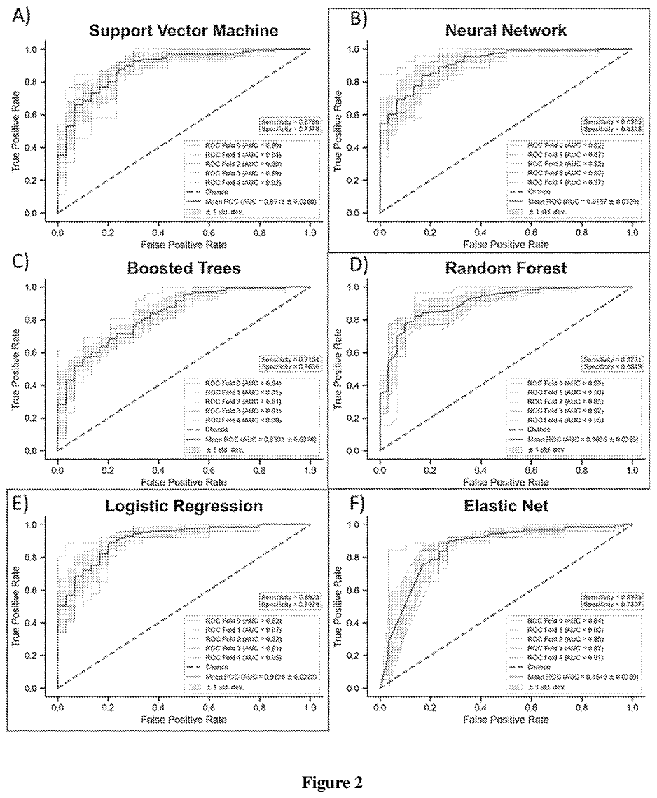

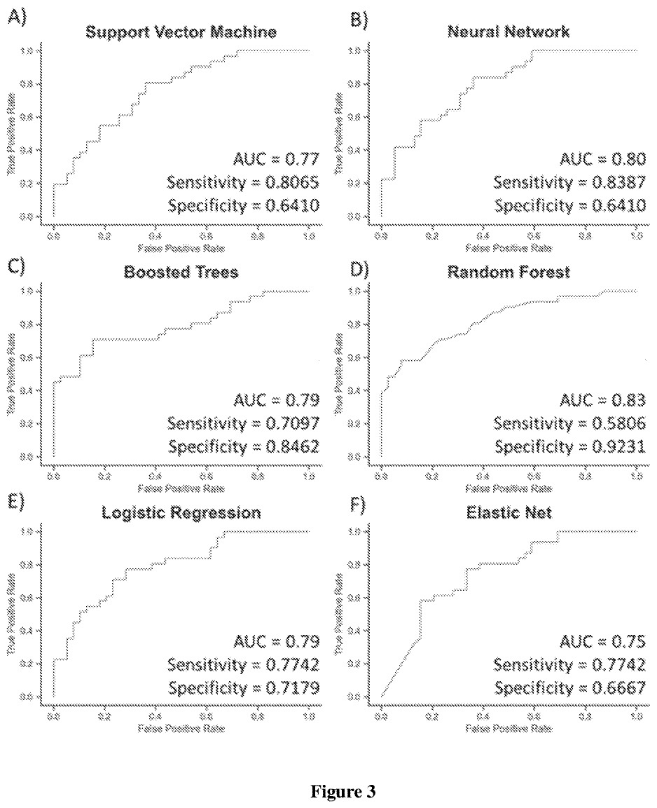

56 pfSNPs and 5 non-genetic factors (age, morning stiffness, hemoglobin value, number of children and platelet count) were selected from approximately 53,000 predictors consisting of a mixture of pfSNPs and 30 non-genetic factors. The selected predictors demonstrated good predictive performance in training set (n=279) as 3 of the 6 models achieved AUC≥ 0.9 (FIG. 2, black box). The ROC curve analysis in training set displayed models achieving cross-validation AUCs between 0.855 and 0.916 (FIG. 2). Predictive capacity of these 61 predictors was further validated in an unseen test set (n=70) when ROC curve analysis showcased models achieving AUCs between 0.751 and 0.826 (FIG. 3).

Interestingly, 51 of the 56 pfSNPs (91%) were eQTLs (expression quantitative trait loci), whose altered expression can be due to the potential alteration of transcription factor, miRNA or ESE/ESS/ISRE binding sites (Table 1). Most of the pfSNPs potentially modulate multiple phenotypes that might lead to the difference in methotrexate response in RA patients.

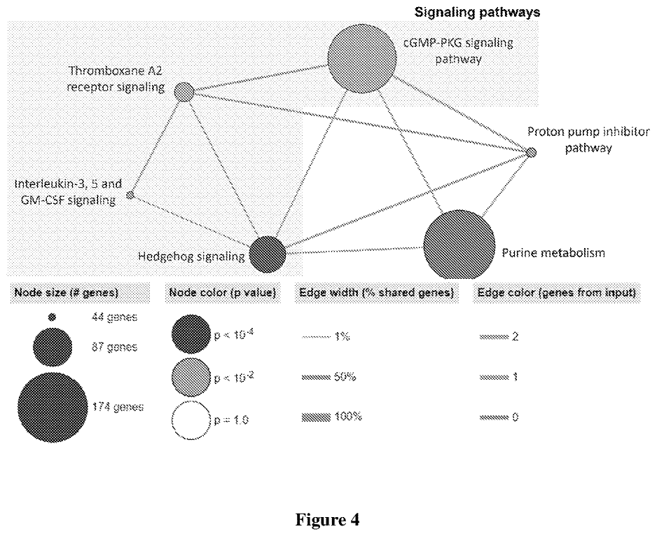

To understand the potential pathways associated with MTX response, pathway analyses were also carried out on the genes associated with the 56 pfSNPs. Genes associated with these 56 pfSNPs were shown to be involved mainly in signaling pathways, proton pump inhibitor pathway and purine metabolism (FIG. 4).

### Haplotypes

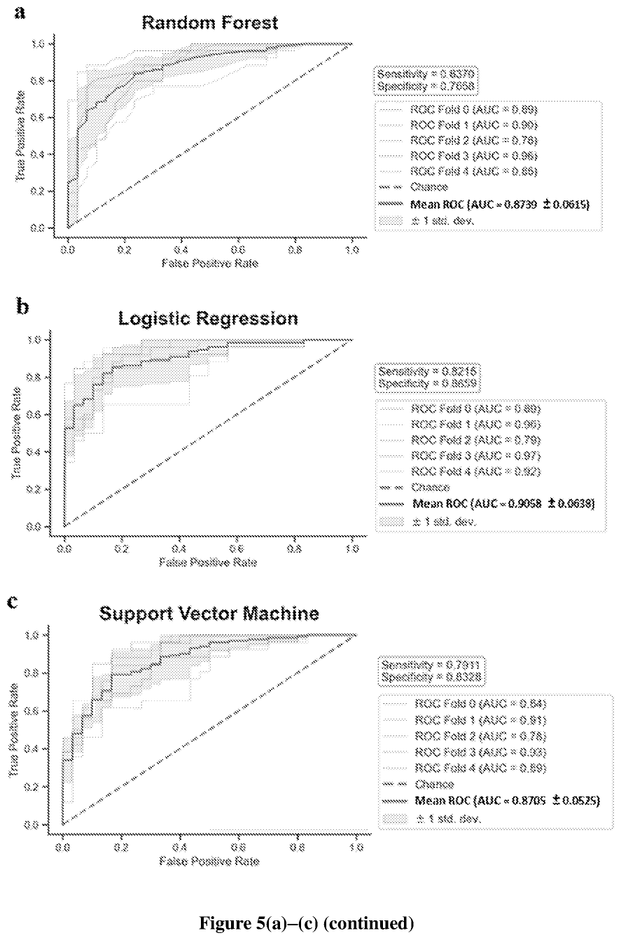

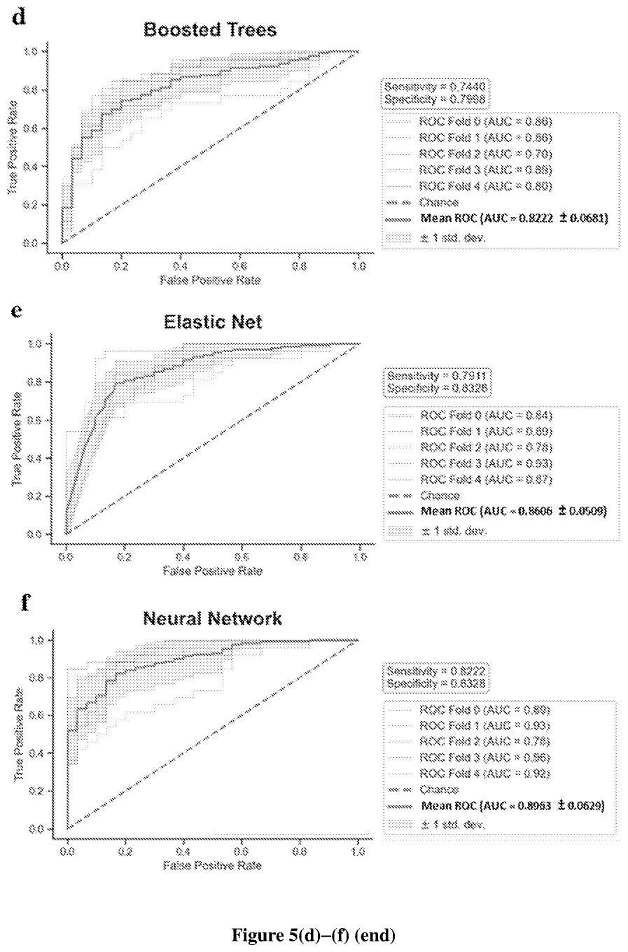

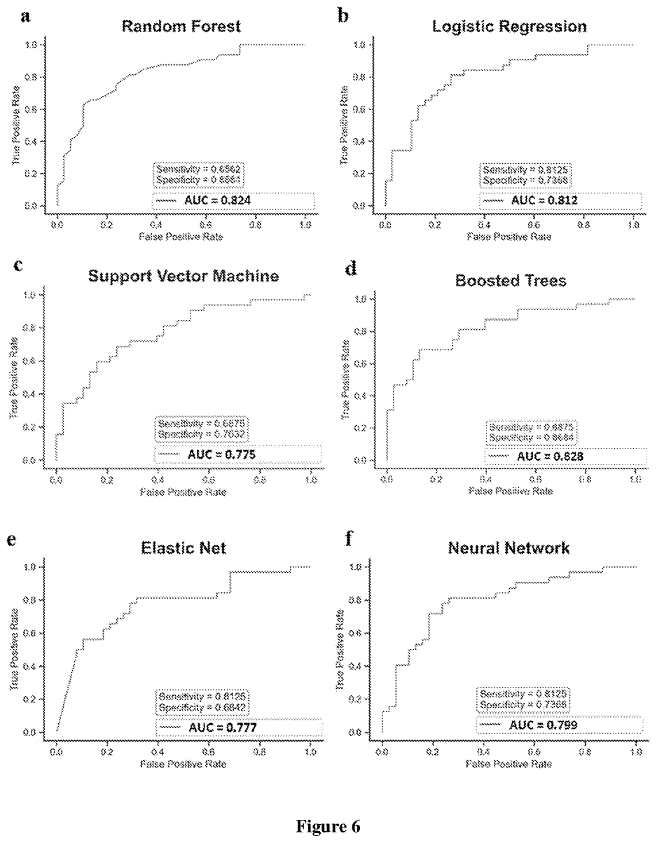

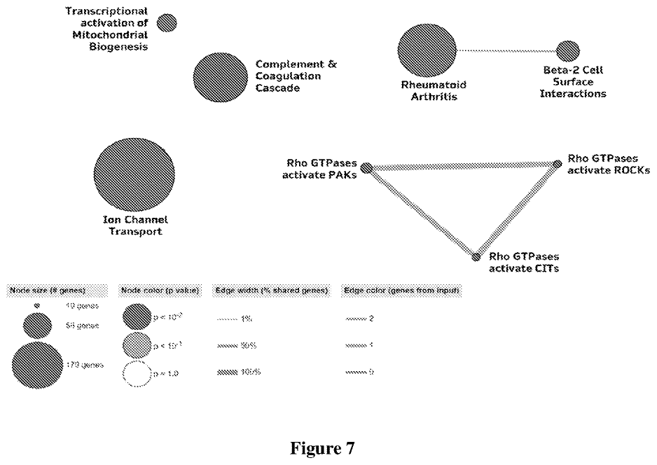

A total of 114,000 SNPs were identified from exome sequencing of blood DNA, which was reduced to 52,331 haplotypes from SNPs in the coding regions of genes (potentially functional coding SNPs, pfcSNPs) derived from ~13,000 genes, representing a ~54.1% reduction in the number of potential predictors. The exclusion of pfcHaps that comprises only reference alleles further reduced the number to 39,160 leading to a ~65.6% reduction in potential predictors, and the remaining predictors were combined with 30 non-genetic factors before performing RFECV. Repeated eight times, RFECV on each training subset reduced the set of 39,160 pfcHaps and identified between 363 to 3612 important features were identified in the eight training subsets important pfcHaps, of which 100 (95 pcfHaps and 5 non-genetic features) were common across all eight training subsets and used for training with cross-validation of the six ML models. The 5 non-genetic features were platelet count, haemoglobin levels, duration of morning stiffness, age, and presence of anti-cyclic citrullinated peptides antibody (anti-CCP). These 100 features were then trained on the six ML models, and exhibited improved predictive performance as compared to using haplotypes alone, with cross-validation AUCs between 0.822 and 0.906, sensitivity between 0.744 and 0.837 and specificity between 0.766 and 0.866 (FIG. 5). As such, these 100 features were noted to be the best predictors and were tested in an unseen test set. Significantly, the robustness of these 100 features to predict MTX in the unseen dataset was evident from the good predictive performance with AUCs between 0.775 and 0.828, sensitivity between 0.656 and 0.813 and specificity between 0.684 and 0.868 (FIG. 6) across all six ML models. To gain mechanistic insights into these 95 predictive pfcHaps, a pathway enrichment analysis was performed. Genes of these pfcHaps are enriched in diverse pathways, including Rho activation of PAKs, ROCKs, and CITs, complement and coagulation cascade, beta-2 cell surface interactions, ion channel transport, transcriptional activation of mitochondrial biogenesis and rheumatoid arthritis.

Of the 100 features determined to be the best predictors, 95 were pfcHaps derived from 142 unique SNPs. These 95 pfcHaps may also be referred to as and may also be referred to as predictive coding haplotypes. 93.0% (132) of these SNPs are eQTL SNPs (Table 2, black box). Approximately 40.8% (58) are non-synonymous while 59.2% (84) are synonymous SNPs. Majority (45) of the non-synonymous SNPs are predicted to be benign, while 5 and 8 SNPs are predicted to be possibly damaging and deleterious, respectively. 18.3% (26) of the 142 SNPs are also predicted to potentially alter transcription factor binding sites while 2.1% (3) can potentially affect miRNA binding sites and 52.1% (74) can potentially modify ESE/ESS sites. The SNPs and their potential functions are summarised in Table 2.

### Example 2

### Methodology

### Study Cohort

This study examines 978 Singaporean RA patients of Chinese ethnicity, who are at least 18 years old, and satisfied the 1987 American College of Rheumatology revised criteria or the 2010 American College of Rheumatology/European League Against Rheumatism criteria for RA. All protocols were performed according to the Declaration of Helsinki and written informed consent was collected from all participants. The study was approved by the National Healthcare Group Domain Specific Review Board (DSRB 2015/00582).

Whole-genome sequencing (WGS) data of 2,732 Singaporean Chinese from the SG10K pilot study served as controls.

### Exome Sequencing, Sequence Alignment, and Quality Control

The exome regions of genomic DNA, collected from peripheral blood mononuclear cells of 978 RA patients, were enriched using the Nimblegen SeqCap EZ kit (Roche). Exomes were captured using the Agilent SureSelect Human All Exon (V5/6) kit (Agilent Technologies, CA), followed by purification using AMPure XP system (Beckman Coulter, Beverly, USA). Quantification was subsequently performed using the Agilent high sensitivity DNA assay on the Agilent Bioanalyzer 2100 system. Whole exome sequencing was performed with Illumina HiSeq 4000 platform with 151 bp pair-end sequencing read.

Training and Test data

978 RA case samples were randomly split into a single training dataset (N=599) and three test sets (N=125/127/127). To maintain the ratio between case and controls, the 2,732 population control samples were similarly split in the same proportion, with a single training dataset (N=1673), and three test sets (N=349/355/355). To ensure that the test cohort is truly ‘unseen’, samples were split into training and test datasets before further downstream analyses/processing.

### Sequence Alignment, Variant Calling, and Quality Control

Utilising the BWA-MEM algorithm (Li, H. et al., Bioinformatics, 25(14):1754-60 (2021)), the sequenced data of RA patients were aligned to the hs37d5 human reference genome, followed by the removal of duplicated reads using PICARD. Each sample was processed separately where realignment, recalibration and genotype calling were performed using the BaseRecalibrator and HaplotypeCaller modules of the Genome Analysis Toolkit (GATK). Using the genomicsDBImport and genotypeGVCF modules to call for variants on samples jointly for the training dataset. For quality control, hard filtering of SNPs was performed based on GATK best practice (QD<2.0, FS >60.0, MQ<40.0, SOR >4.0, MQRankSum <−12.5, ReadPosRankSum <−8.0) using the VariantFiltration module.

### Pre-Processing of Training Dataset

Training dataset of both cases (N=599) and controls (N=1673) were merged together (N=2272) using BCFtools (Danecek, P. et al., Gigascience, 10(2):1-4 (2021)) to identify SNPs that are common in both case and control datasets. The merged training dataset was further processed by removing SNPs with minor allele frequency <1%, or >10% genotype missingness or deviate from Hardy-Weinberg equilibrium (p-value <0.01).

Missing genotypes were phased and imputed using the Beagle 5.1 software with HapMap Phase II recombination maps and 1000 Genomes Project phase III reference panels for each respective chromosome (Browning, S. R. et al., Am J Hum Genet, 81(5):1084-97 (2007)). Thereafter, a Bayesian Ridge model, coupled with the IterativeImputer function from the Scikit-learn Python module (Pedregosa, F. et al., Journal of Machine Learning Research, Vol. 12 (2011)), was fitted using the training dataset. This fitted model was then used to impute the remaining unimputed genotypes in the training dataset.

Selecting Features/SNPs that are Predictive for RA Cases

Within the training set, features identified to have the same genotype across >90% of the samples were excluded from subsequent analyses. Additionally, for features sharing a >95% correlation (Pearson Correlation Coefficient) in each chromosome, only one of the correlated features was retained for further analyses. The remaining training dataset of 76,713 SNPs was then processed into eight subsets of variable sample sizes using a stratified random sampling with replacement approach to ensure that features selected are stable. Thereafter, the recursive feature elimination with cross-validation (RFECV) algorithm was implemented with a Random Forest classifier estimator using the Scikit-learn Python module to identify an optimal set of important features sufficient for the prediction for each training subset. With the goal of obtaining features with a high stability of importance, features commonly selected across all eight subsets were chosen as the final set of features for further evaluation of predictive performances.

### Extraction of Genotypes for Selected SNPs for the Test Datasets

Using BCFtools, the genotype data for RA case samples in test sets were identified for the selected SNPs (after training) directly from the GVCF files produced from the HaplotypeCaller step. Separately, genotype data for population control samples were extracted from the VCF files obtained from the SG10K Pilot Study. For each of the 3 unseen test datasets, genotype data from the RA case and population control samples were combined. Thereafter, a Bayesian Ridge model, coupled with the IterativeImputer function from the Scikit-learn Python module, was fitted using the training dataset. The fitted model was subsequently used for the imputation of any missing genotypes in each of the 3 unseen test datasets. The individual test datasets consisting of both RA cases and population control samples were then independently used to evaluate the predictive performance of models that were trained using the train dataset.

### Evaluating the Predictive Performance of Selected Features Using Supervised ML

The selected features were assessed across five diverse ML classifiers: Logistic Regression, Support Vector Machines, Naïve Bayes, Random Forest and XGBoost. Within the training dataset, a 5-fold cross-validation using stratified k-fold was performed for each of the five classifiers. Evaluation of predictive performance was conducted by referencing metrics such as the area under the curve (AUC) of a receiver operating characteristic (ROC) curve, sensitivity, specificity, accuracy scores, and average precision (area under a precision-recall curve). Similarly, the five classifiers were also fitted with the entire training datasets composed of the selected features and tested against the three independent unseen test datasets for their predictive performances based on the same metrics. The selected features were ranked based on their mean feature importance scores provided by the Random Forest estimator used in RFECV. To identify the minimum number of features required to achieve an optimal performance metrics (namely AUC, sensitivity, and specificity), the selected features were added one at a time to train models and evaluated for their predictive performance. The Shapley Additive explanations (SHAP) method (Lundberg, S. M. et al., Adv Neural Inf Process Syst, 2017-December:4766-75) was adopted to explore the contribution of the selected features in the machine learning models for the classification of RA case samples, focusing on the Random Forest classifier that was initially used for the selection of predictive features.

To verify that the observed predictive performances by the selected features are not a random occurrence, the same number of features were randomly sampled (with replacement) from all the features in the training dataset prior to performing RFECV. These randomly sampled features were evaluated across all three unseen test sets and the results were used to plot a distribution of model performances using the ROC-AUC metric, across 1,000 iterations of sampling.

### Annotation and Analysis of Potential Variant Functions

Selected SNPs were annotated using ANNOVAR (Wang, K. et al., Nucleic Acids Research, Volume 38, Issue 16, 1 Sep. 2010) and SNPNexus database (Oscanoa, J. et al., Nucleic Acids Res, 48(W1):W185-92 (2020)), to identify their corresponding functional regions and genes. To obtain information regarding their potential functionality (e.g., transcription factor binding sites, miRNA binding sites, exonic splice enhancer/silencer (ESE/ESS), etc), these SNPs were referenced against the pfSNP database resource (Wang, J. et al., Hum Mutat. 2011 January, 32(1):19-24 (2011)), which has been updated to include information such as expression-associated SNPs or expression quantitative trait loci (eQTLs) (Võsa, U. et al., BioRxiv 44736 (2018), Lonsdale, J. et al., Nat Genet, 45:580-5 (2013)). SNPs that were not predicted to be potentially functional were further interrogated for neighbouring pfSNPs in linkage disequilibrium (LD) (R2 >0.8). The WGS data from the SG10K pilot study was used to identify pfSNPs in LD with these selected SNPs.

### Machine-Learning-Optimized Polygenic Risk Scores (PRS)

To improve interpretability and clinical applicability of the identified predictive SNPs for individual patients, a PRS was developed based on the ML-identified predictive SNPs. The effect sizes of these predictive SNPs were determined through univariate logistic regression analyses using PLINK (Purcell, S. et al., Am J Hum Genet, 81:559-75 (2017)), assuming additive effects of allele dosage, of all the samples in the training dataset. The following is the formula for calculating the PRS based on our 9 SNPs (Choi, S. W. et al., Nat Protoc, 15(9):2759-72 (2020)):

$$PRS = \frac{{\sum }_{i}^{9} {S}_{i} \times {G}_{ij}}{P \times {M}_{j}}$$

For each SNP i within a sample j, the product of the SNP's effect size (Sᵢ) and the sample's allelic dosage (Gij) was calculated. The resultant product for all selected SNPs were then summed and divided by the product of the ploidy (P) of an individual (2 for humans) and the number of non-missing variants in that sample (Mⱼ). The resultant PRS takes into consideration the possibility of missing genotypes by identifying the average PRS through the division of the number of non-missing SNP dosages. Most importantly, it prevents PRS of samples with missing genotypes to be consistently lower than those with complete data of their genotypes, mitigating bias of these samples towards a lower risk (Collister, J. A., Front Genet., 2022 Feb. 18; 13:105). The distribution of PRS of samples in the training set was then plotted.

The same effect sizes of SNPs established from the training set was similarly used to calculate the PRS of samples across the 3 unseen test sets. Logistic regression was performed to examine the significance of association between the calculated PRS with RA. Using PRS as the sole predictor in our ML models, we further assessed the predictive capacity of PRS for RA.

### Results

### Case and Population Control Datasets are Comparable

Exome sequencing was performed on Singaporean Chinese RA case samples, while WGS data of Singaporean Chinese population controls were obtained from the SG10K pilot study. As data from cases and controls were derived from different sequencing platforms, principal component analyses (PCA) were performed to establish that there was no batch effect that could confound our analysis.

a Signature of 9 SNPs was Identified that Robustly Classifies RA in 3 Independent Unseen Datasets

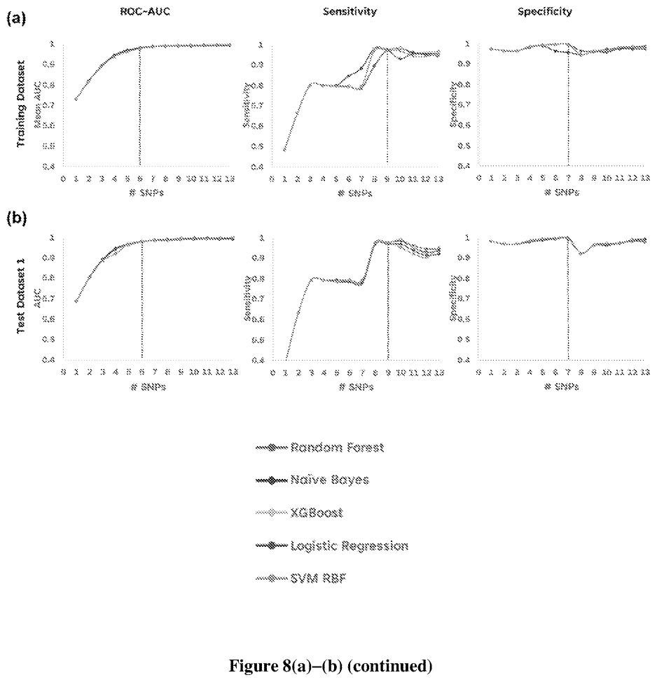

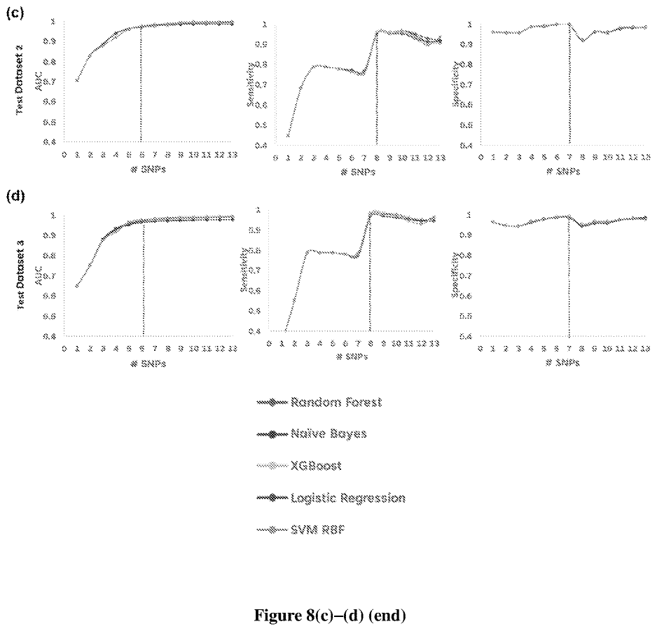

To reduce dimensionality and identify a robust set of SNPs that are resistant to sample size bias, feature selection using the RFECV algorithm was employed on eight randomly generated variable-sized sample subsets. Thirteen SNP features, with mean feature importance scores between 0.0118 to 0.0612 were commonly identified across all 8 subsets. To identify the minimum number of features necessary for optimal predictive performance, stepwise inclusion of each of the 13 SNPs based on their feature importance scores (Tables 3 and 4) was assessed through cross-validation in the training dataset across all 5 ML models as well as the 3 independent unseen test datasets. As shown in FIG. 8, 9 out of the 13 SNPs (reduction of 30%) was required to achieve a reasonable predictive performance in all the 3 metrics examined (>90% for AUC, sensitivity, and specificity) across both the training as well as 3 independent unseen test datasets. While 8 SNPs were sufficient to achieve reasonably good AUC and sensitivity, the addition of the 8th SNP resulted in a dip in the specificity, hence 9 SNPs is an optimal number to achieve high performance in both sensitivity and specificity, in addition to AUC.

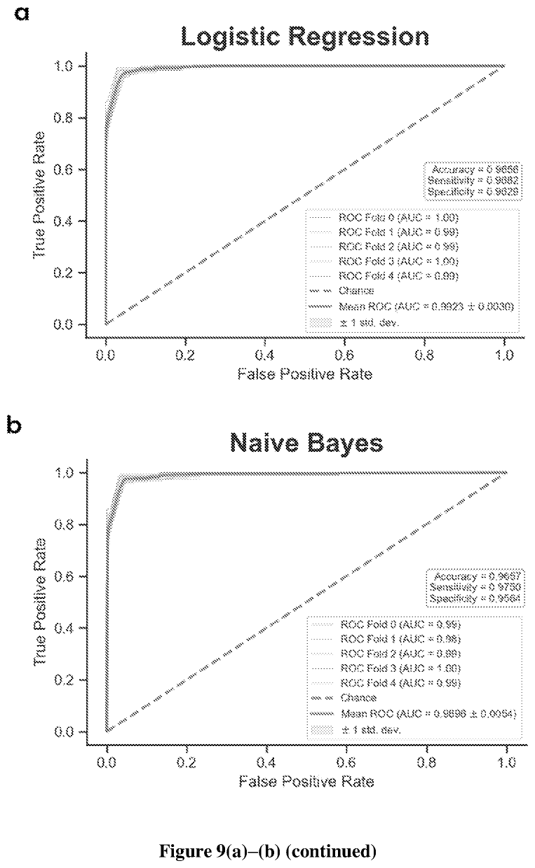

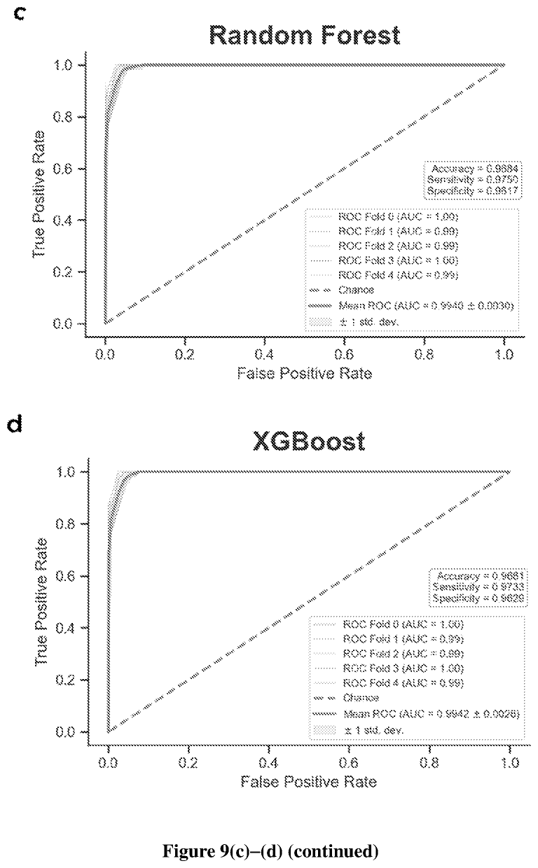

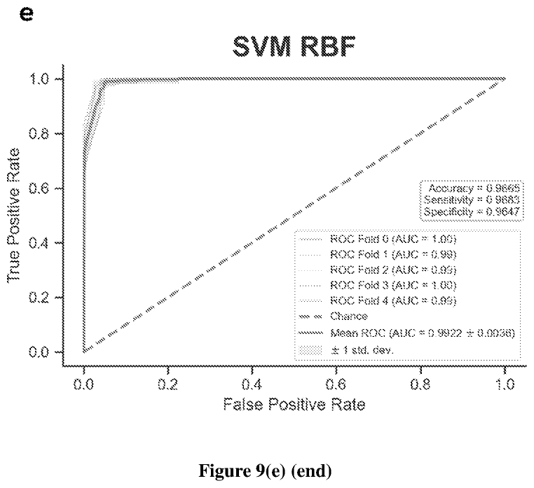

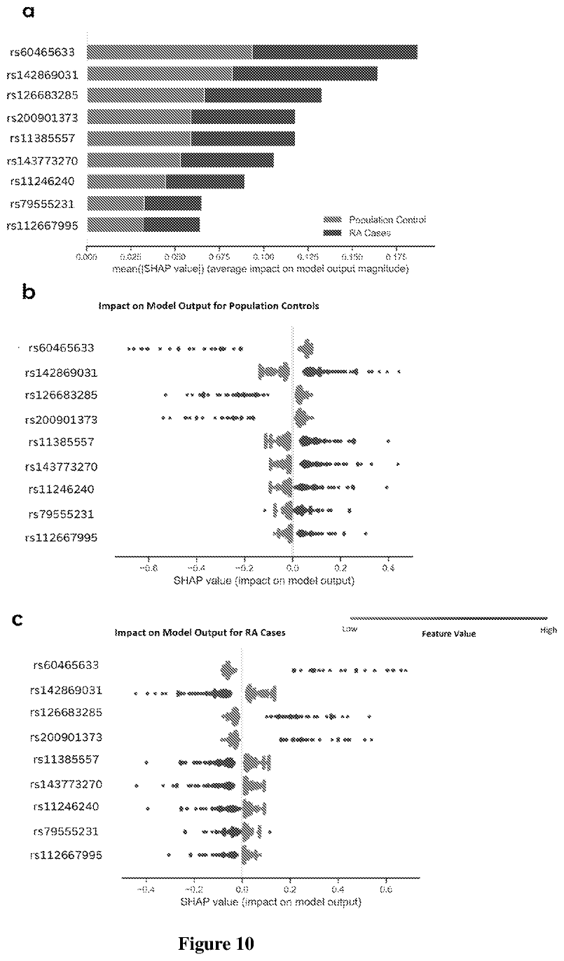

These 9 SNPs achieved mean AUC values between 0.990-0.994 when assessed using Cross-Validation in the Training dataset across all 5 selected ML models (Table 5, FIG. 9). Significantly, when tested against 3 independent unseen datasets, these same 9 SNPs performed exceptionally well, with AUC >0.97 and all other pertinent metrics (F1 Score, Accuracy, Sensitivity, Specificity, and Average Precision) above 0.90 in all the different ML models (Table 6). SHAP analyses of the 9 selected SNPs within the Random Forest classifier model (FIG. 10) reveals the contribution of each of the SNPs towards the model prediction output, ordered from the SNPs with the greatest contribution to the least amongst the 9 SNPs.

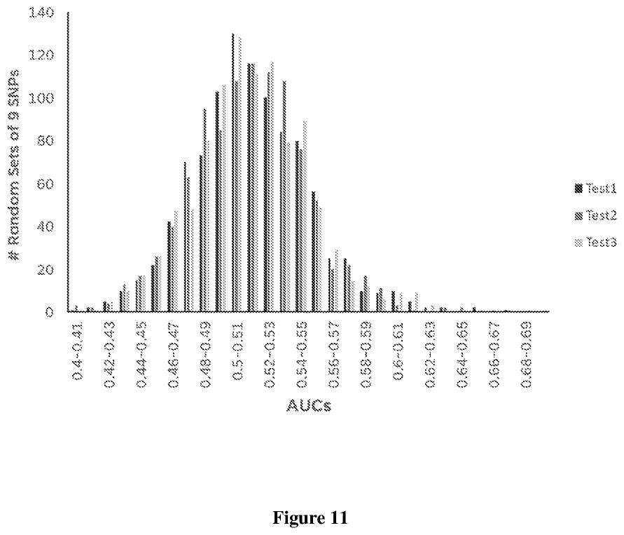

With such excellent predictive performance in 3 different unseen datasets, it is pertinent to evaluate the validity of the observation and give assurance that the excellent predictive performance is not merely due to random chance. A thousand iterations of random sampling of 9 SNPs from the total pool of >70,000 SNPs were performed. These randomly selected 9 SNPs were then evaluated, as above, for predictive performance using Random Forest, one of the 5 ML models, in the 3 unseen Test dataset. AUCs obtained were then binned with intervals of 0.01 and the distribution of AUCs were plotted. As evident in FIG. 11, the AUCs of the 1000 randomly identified 9 SNPs are normally distributed with peak AUC between 0.50-0.51 and the highest AUC is less than 0.7.

PRS Utilising 9 ML-Identified Predictive SNPs Clearly Distinguishes RA Patients from Healthy Individuals

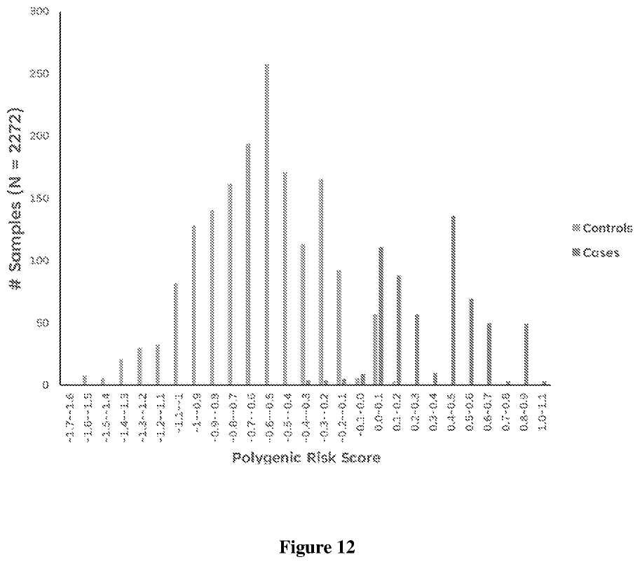

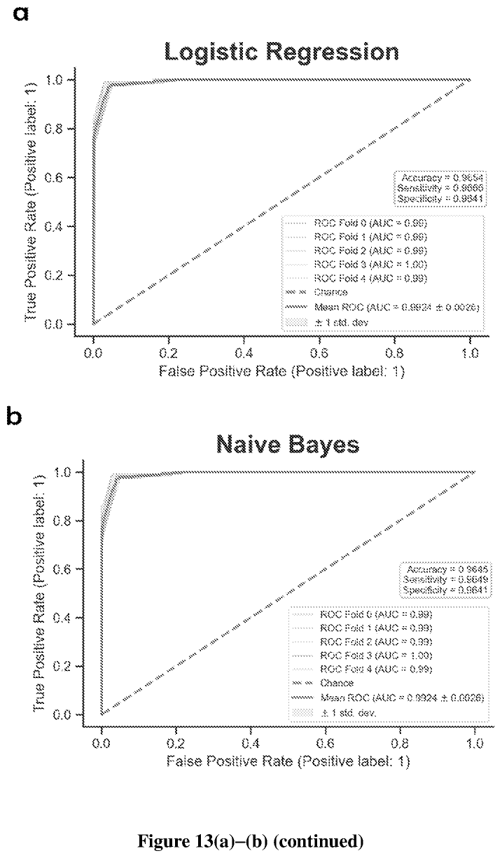

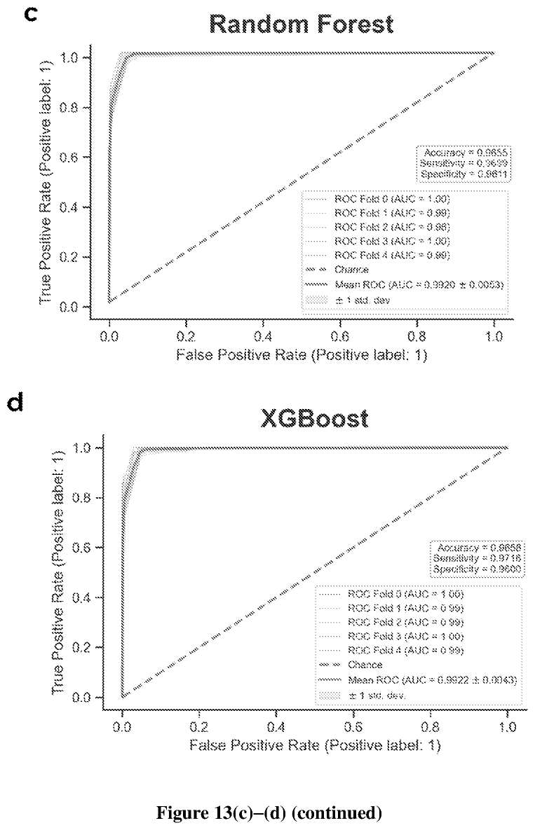

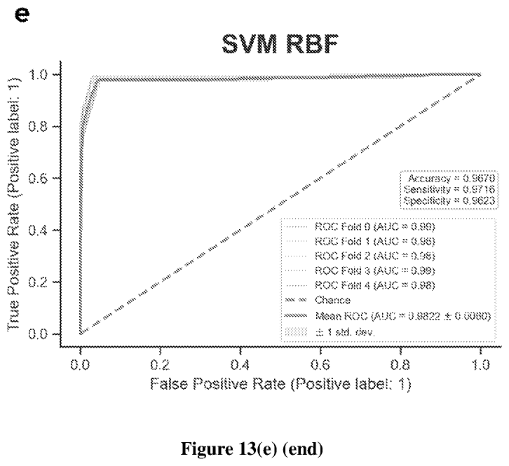

Univariate logistic regression analyses revealed that all 9 ML-identified predictive SNPs were significantly associated with RA (P<1×10⁻⁵) (Table 3). Six ML-identified predictive SNPs have effect sizes in the negative range (β: −5.24 to −2.23) and hence confer protection against RA while the remaining three SNPs with positive effect sizes (β: 3.50 to 7.28) predispose to RA. The ML-optimized PRS of training set samples from RA patients and control population displayed relatively normal but distinct distributions with some overlap (−0.3 to 0.2), with PRS of RA patients ranging from −0.3 to 1.1, while PRS of control population ranges from −1.7 to 0.2 (FIG. 12). Notably, using logistic regression analyses, PRS was found to be significantly associated (P<1×10⁻⁶) across all 3 unseen test sets. Significantly, the predictive performance of the sole ML-optimized PRS (Table 7, FIG. 13) was found to be comparable to ML-identified 9 SNPs (Tables 5 and 6) across all 5 selected ML models and 3 independent test sets.

Characteristics of these 9 Predictive SNPs

Although these 9 SNPs were identified primarily from exome sequenced DNA, the majority (6, 67%) of these SNPs are intronic, while 3 (33%) reside within exons (Table 3). The 3 exonic SNPs were all non-synonymous, with one predicted to be a deleterious alteration. Two of the intronic predictive SNPs are potential eQTL (expression quantitative trait locus) SNPs, predicted to be associated with changes in expression (Table 3). Of the other 4 predictive intronic SNPs, 3 SNPs are in strong linkage disequilibrium (LD) (R2>0.8) with SNPs that are potentially functional.

To gain further insight into the significance of these predictive SNPs, several GWAS databases were interrogated to determine if any of these SNPs were previously reported to be significantly associated with any phenotype. Three SNPs were identified by 3 GWAS databases—BioBank Japan PheWeb (Sakaue, S. et al., Nat Genet, 53(10):1415-24 (2021)), IEU Open GWAS Project (Elsworth, B. et al., bioRxiv, 2020.08.10.244293 (2020)), GWAS Atlas (Tian, D. et al., Nucleic Acids Res, 48(D1):D927-32 (2020))—to be significantly (P<1×10⁻⁵) associated with various phenotypes. These phenotypes were summarized into 7 general categories (Table 3).

The 9 SNPs reside in 10 different genes with one residing in the exonic regions of 2 genes. These genes reside in pathways such as signal transduction, sensory perception, immune, and metabolism of lipids/proteins (Table 3). Amongst these pathways, some such as signal transduction, sensory perception, immune, metabolism of lipids/proteins are consistent with characteristics of pathology/development of RA. Two of these genes have previously been reported to be associated with RA (Table 3).

### Discussion

In this study, through rigorous ML feature selection that is tolerant to differences in sample sizes, a signature of 9 SNPs was identified that can predict RA with excellent predictive performance not only in the training dataset (mean AUC >0.99; mean sensitivity >0.96, mean specificity >0.95, mean accuracy >0.96, and mean average precision >0.96) assessed through cross-validation, but also in not one, but 3 independent, unseen test datasets (AUC >0.97; sensitivity >0.91, specificity >0.95, F1 score>0.90, accuracy >0.94, and average precision >0.93 in all 3 test datasets). This excellent predictive performance is unlikely due to random chance since the predictive performance of 1,000 set of 9 randomly-selected SNPs was poor with AUC<0.7, and majority of the 9 randomly-selected SNPs have AUC of only between 0.50-0.51.

To facilitate interpretability and clinical applicability of the 9 ML-identified predictive SNPs for individual patients, a PRS was developed based on these 9 ML-identified predictive SNPs. Notably, not only are the 9 ML-identified predictive SNPs significantly (P<1×10⁻⁵) associated with RA individually, the calculated PRS score (from these 9 SNPs) were also found through logistic regression to be significantly (P<1×10⁻⁶) associated with RA across all 3 unseen datasets and this single ML-optimized PRS was also found to have comparable excellent predictive performance as the 9 SNPs across all 5 selected ML models and 3 independent test sets.

Although exome sequencing data was interrogated, only a minority (3 out of 9) of the predictive SNPs reside in the coding region. Two (rs1266832853 and rs60465633) of the 3 coding predictive SNPs are benign, non-synonymous susceptibility SNPs with positive effect sizes while one (rs143773270) is a potential deleterious, non-synonymous protective SNP with negative effect size. The majority of the predictive SNPs resides in the introns (6 out of 9). Most (5 out of 6) of these intronic predictive SNPs are potentially protective SNPs with negative effect sizes. These potentially protective predictive SNPs in non-coding regions with negative effect sizes, are potential eQTL SNPs or are in strong LD (R²>0.8) with non-interrogated SNPs that are predicted to be potentially functional, modulating expression of the gene by being eQTL SNPs, or potentially altering transcription factor binding sites (TFBS) or intronic splicing regulatory elements (ISRE). The putative functionality of the sole intronic predictive susceptibility SNP (rs200901373) with positive effect size remains unknown. These data thus suggest that while the majority (2 out of 3) of the non-synonymous, predictive coding SNPs have positive effect size and thus may confer susceptibility to RA through modulating protein structure/function, the majority of the (5 out of 6) of intronic predictive SNPs have negative effect size and thus may confer protection against RA through modulating the expression of the intricate network of genes as these intronic predictive SNPs are either eQTL SNPs or have potential to alter TFBS/ISRE sites.

None of the 9 predictive SNPs have previously been reported to be associated with RA. To gain further insight into these 9 predictive SNPs, numerous GWAS databases were interrogated to evaluate if any of these 9 predictive SNPs were previously reported via GWAS to be associated with any disease/phenotype/function which may help explain the role of these SNPs/genes in RA. As GWAS mainly interrogate tag-SNPs and this study interrogates exomic SNPs, only 3 of these 9 predictive SNPs were reported by 3 GWAS databases (BioBank Japan PheWeb, IEU Open GWAS Project, GWAS Atlas) to be significantly associated (P<1×10⁻⁵) with several different functions (including eQTL association) and some diseases, which can be categorized into 6 different themes (Table 3). Notably, most of these association were consistent with the characteristics or phenotype of RA. For example, rs11385557 in the intronic region of BAIAP2L1 was found in OpenGWAS database to be significantly (P<7.69×10⁻⁵) associated with peripheral nerve disorders which is consistent with RA patients often experiencing peripheral neuropathy with pain, numbness, and muscle weakness. Similarly, rs11385557 was also reported by OpenGWAS database to be significantly (P<9.08×10⁻⁵) associated with lymphocyte and monocyte counts. This is consistent with reports of lymphopenia (low lymphocyte counts) commonly observed in RA patients, as well as monocytes activation and migration into joints in early RA.

Although these 9 SNPs were not previously associated with RA, 2 of them reside in disease susceptibility genes (Table 3, Row 5 & 9). rs11385557 is found in the intron of the BAIAP2L1, an insulin receptor tyrosine kinase gene (Table 3). The expression of the BAIAP2L1 gene is positively correlated with C-reactive protein (CRP) levels within fibroblast-like synovial cells from RA patients. CRP is an immune regulator that is commonly used as a marker for systemic inflammation in RA. Although the intronic SNP was not predicted to be potentially functional, it was found to be in strong linkage disequilibrium (r²>0.8) with 19 potentially functional SNPs, most of which are associated with modulation of gene expression (eQTL SNPs), while some are predicted to alter intronic splice regulatory elements (ISRE). Hence, the above observation is consistent with SNPs in LD with rs11385557 modulating gene expression of the BAIAP2L1 gene, which in turn alters the CRP levels in RA patients. rs112667995 resides within the intron of the PRKN (Parkin RBR E3 Ubiquitin Protein Ligase) gene. PRKN deficiency ameliorates inflammatory arthritis through the suppression of p53 degradation). Similarly, although this intronic SNP (rs79555231) is not predicted to be potentially functional (Table 3), it is in strong LD (r²>0.8) with 21 potentially functional SNPs that are mainly predicted to alter transcription factor binding sites (TFBS). Thus, SNPs in LD with rs79555231 may influence the expression of PRKN which in turn modulates inflammatory arthritis. Taken together, both these intronic SNPs are in LD with markers that modulate gene expression of either BAIAP2L1 or PRKN that is associated with RA. Since majority of the predictive SNPs are in non-coding regions and many of the potentially functional SNPs likely reside beyond the regions that were sequenced through exome sequencing, it may thus be worthwhile to build models from WGS data.

These 9 SNPs have potential to be clinically applicable for diagnosing individual patients with RA through the development of rapid genotyping assays for these SNPs. With improvements of WGS technology coupled with its rapidly declining cost, it will not be surprising that most, if not all, individuals will have their genome sequenced in the foreseeable future. Then, it will be cost-effective to deploy PRS on a large scale. However, due to resource constraints, current WGS data is primarily stored as variant call format (VCF) files with small data storage space requirements. One limitation of storing WGS data as VCF is that, in order to generate VCF files, raw sequences from a group of individuals are aligned and variants are then identified based on a reference genome. The sequence identity of loci which do not show variability between the group of individuals and the reference genome are not stored. As such, depending on the size of the group of individuals, the number of variations stored will be different, with more variation stored in VCF files of large and more diverse group and less variation stored in VCF files of smaller and more homogenous group of individuals. For locus without sequence identity assigned, it is not possible to accurately extract the genotype information at the individual level. One cannot assume that locus to be the homozygous genotype of the reference genome as the unassigned region could also be excluded due to low mapping quality or poor read coverage during sequencing. Hence, for WGS data to be clinically applicable at an individual level, an alternative VCF file, the genomic VCF (gVCF) file format, which can be generated using the GATK suite, could be explored. Although the storage size required for gVCF files is larger than typical VCF files, it is still overall much smaller compared to the storage of BAM files containing the sequencing read alignments. The advantage of the gVCF file is that it stores not only information of the genotypes of variant site, but it also compactly stores information of the invariant genomic regions, facilitating the more accurate assignment of genotype at invariant sites for clinical implementation of our prediction models.

TABLE 1
Functional characteristics of 56 pfSNPs that are predictive of MTX response:

| | | | | Aggregate | | | | ESE/ESS | Coding | | Codon | |
|---|---|---|---|---|---|---|---|---|---|---|---|---|
| | | Gene | Gene | Feature | Expression | TF | miRNA | or | snps | AA | Use | AA |
| No | RSID | name | location | Importance | change | binding | binding | ISRE | type | Effect | Diff | change |
| 1 | rs10221770 | DDX1 | UTR5 | 0.011 | Y | + | •• | •• | •• | •• | •• | •• |
| 2 | rs2286362 | ADGRE2 | UTR5 | 0.006 | Y | •• | ? | •• | •• | •• | •• | •• |
| 3 | rs1056538 | ICAM5 | Exonic | 0.006 | Y | | •• | E | N | B | •• | V > I |
| 4 | rs35618062 | ZNF488 | Exonic | 0.006 | Y | •• | •• | E | N | D | •• | A > V |
| 5 | rs11618506 | ERICH6B | Exonic | 0.010 | Y | •• | •• | E | N | B | •• | L > P |
| 6 | rs560096 | IGHMBP2 | Exonic | 0.019 | Y | •• | •• | E | N | B | •• | L > S |
| 7 | rs7872034 | SMC2 | Exonic | 0.033 | Y | ? | •• | E | S | •• | •• | L > L |
| 8 | rs3752293 | FAM83D | Exonic | 0.008 | Y | •• | •• | E | S | •• | | L > L |
| 9 | rs12305038 | PDE3A | Exonic | 0.010 | •• | | •• | E | N | B | •• | D > N |
| 10 | rs9358856 | CARMIL1 | Exonic | 0.007 | Y | •• | •• | •• | N | B | •• | V > I |
| 11 | rs73118352 | C12orf56 | Exonic | 0.020 | Y | •• | •• | •• | N | P | •• | D > N |
| 12 | rs2487302 | KIF26A | Exonic | 0.009 | Y | ? | •• | •• | S | •• | •• | L > L |
| 13 | rs12347076 | OR13D1 | Exonic | 0.010 | Y | | •• | •• | S | •• | •• | L > L |
| 14 | rs325400 | MEF2A | Exonic | 0.007 | Y | | •• | •• | S | •• | •• | G > G |
| 15 | rs1557567 | ZNF76 | Exonic | 0.007 | Y | •• | •• | E | S | •• | •• | H > H |
| 16 | rs2240735 | ADCY9 | Exonic | 0.007 | Y | •• | •• | E | S | •• | •• | A > A |
| 17 | rs6116 | SERPINA5 | Exonic | 0.007 | Y | •• | •• | E | S | •• | •• | I > I |
| 18 | rs11146986 | POLE | Exonic | 0.007 | •• | ? | •• | E | S | •• | •• | A > A |
| 19 | rs13293151 | ADAMTSL1 | Exonic | 0.002 | •• | ? | •• | E | S | •• | •• | T > T |
| 20 | rs929387 | GLI3 | Exonic | 0.008 | •• | •• | •• | •• | N | D | •• | P > L |
| 21 | rs8192646 | TAAR2 | Exonic | 0.008 | Y | •• | •• | •• | N | •• | •• | W > X |
| 22 | rs10412915 | NLRP2 | Exonic | 0.008 | Y | •• | •• | •• | S | •• | •• | F > F |
| 23 | rs2232103 | URGCP | Exonic | 0.008 | Y | •• | •• | •• | S | •• | •• | H > H |
| 24 | rs2236527 | MTG2 | Exonic | 0.015 | Y | •• | •• | •• | S | •• | •• | F > F |
| 25 | rs34968651 | PTGDR | Exonic | 0.015 | Y | •• | •• | •• | S | •• | •• | G > G |
| 26 | rs3796099 | ANKRD53 | Exonic | 0.015 | Y | •• | •• | •• | S | | •• | T > T |
| 27 | rs7722287 | SLC12A7 | Exonic | 0.016 | Y | •• | •• | •• | S | •• | •• | Y > Y |
| 28 | rs28446896 | MGAM | Intronic | 0.009 | Y | •• | •• | I | •• | •• | •• | •• |
| 29 | rs2853445 | PRSS56 | Intronic | 0.026 | Y | •• | •• | I | •• | •• | •• | •• |
| 30 | rs4681297 | PLOD2 | Intronic | 0.010 | Y | •• | •• | I | •• | •• | •• | •• |
| 31 | rs4796601 | HAP1 | Intronic | 0.018 | Y | •• | •• | I | •• | •• | •• | •• |
| 32 | rs8191489 | APRT | Intronic | 0.004 | Y | •• | •• | I | •• | •• | •• | •• |
| 33 | rs881863 | FAM26E; | Intronic | 0.009 | Y | •• | •• | I | •• | •• | •• | •• |
| | | TRAPPC3L | | | | | | | | | | |
| 34 | rs9371061 | RNF144B | Intronic | 0.017 | Y | •• | •• | I | •• | •• | •• | •• |
| 35 | rs2777729 | DDX58 | Intronic | 0.007 | Y | + | •• | •• | •• | •• | •• | •• |
| 36 | rs474242 | MROH2A | Intronic | 0.010 | Y | + | •• | •• | •• | •• | •• | •• |
| 37 | rs4850901 | EIF5B | Intronic | 0.009 | Y | | •• | •• | •• | •• | •• | •• |
| 38 | rs11808959 | CACHD1 | Intronic | 0.028 | Y | •• | •• | •• | •• | •• | •• | •••• |
| 39 | rs2072713 | CSF2RB | Intronic | 0.012 | Y | •• | •• | •• | •• | •• | •• | •••• |
| 40 | rs2271508 | NUP210 | Intronic | 0.006 | Y | •• | •• | •• | •• | •• | •• | •• |
| 41 | rs2286342 | EVC | Intronic | 0.013 | Y | •• | •• | •• | •• | •• | •• | •••• |
| 42 | rs2286874 | MYO1C | Intronic | 0.016 | Y | •• | •• | •• | •• | •• | •• | •••• |
| 43 | rs28507496 | OBP2B | Intronic | 0.008 | Y | •• | •• | •• | •• | •• | •• | •••• |
| 44 | rs3730353 | FYN | Intronic | 0.009 | Y | •• | •• | •• | •• | •• | •• | •••• |
| 45 | rs4821704 | TRIOBP | Intronic | 0.009 | Y | •• | •• | •• | •• | •• | •• | •••• |
| 46 | rs77273876 | FCAR | Intronic | 0.008 | Y | •• | •• | •• | •• | •• | •• | •••• |
| 47 | rs8039777 | ULK3 | Intronic | 0.016 | Y | •• | •• | •• | •• | •• | •• | •••• |
| 48 | rs11983326 | ABCB5 | Intronic | 0.015 | •• | | •• | •• | •• | •• | •• | •• |
| 49 | rs3802482 | ZDHHC21 | UTR3 | 0.006 | Y | + | +− | •• | •• | •• | •• | •• |
| 50 | rs15881 | GFRA2 | UTR3 | 0.015 | Y | •• | •• | E | •• | •• | •• | •••• |
| 51 | rs62145930 | WDR92 | UTR3 | 0.010 | Y | •• | •• | •• | •••• | •• | •• | •• |
| 52 | rs879620 | ADCY9 | UTR3 | 0.015 | Y | •• | •• | •• | •• | •• | •• | •• |
| 53 | rs4412304 | FAM185BP | ncRNA_exonic | 0.007 | Y | + | •• | •• | •• | •• | •• | •• |
| 54 | rs2168518 | MIR4513 | ncRNA_exonic | 0.009 | Y | •• | ? | •• | •• | •• | •• | •• |
| 55 | rs1291362 | HTR7P1 | ncRNA_exonic | 0.021 | Y | •• | •• | •• | •• | •• | •• | •• |
| 56 | rs165704 | BMS1P20; | Intergenic | 0.013 | Y | •• | •• | •• | •• | •• | •• | •• |
| | | ZNF280B | | | | | | | | | | |

Legend:
Y Yes
N Nonsynonymous SNV
S Synonymous SNV
+ Create TF/miRNA binding site
− Delete TF/miRNA binding site
? Unknown effect
E ESE/ESS
I ISRE
B Benign effect on AA
P Possibly damaging effect on AA
D Deleterious effect on AA

TABLE 2
142 SNPs in 95 coding haplotypes as predictors of methotrexate response

| | | | | | | Aggregate |
|---|---|---|---|---|---|---|
| | | | | | | Feature |
| | | | | | Aggregate | Importance |
| | | | | | Feature | Score |
| No. | RSID | SNPID | Gene | Haplotype | Importance | Ranking |
| 1 | rs4617548 | 11:16133413:A:G | SOX6 | SOX6_1.0 | 0.034869 | 4 |
| 2 | rs3731986 | 2:172216969:T:C | METTL8 | METTL8_0.0 | 0.031633 | 5 |
| 3 | rs2279348 | 16:89350038:G:A | ANKRD11 | ANKRD11_2.0 | 0.023206 | 6 |
| 4 | rs7935 | 19:11105608:T:C | SMARCA4 | SMARCA4_3.0 | 0.022074 | 7 |
| 5 | rs9332464 | 5:74921686:G:A | ANKDD1B | ANKDD1B_3.0 | 0.021949 | 8 |
| 6 | rs10929378 | 2:15747393:C:T | DDX1 | DDX1_0.0 | 0.018593 | 10 |
| 7 | rs2014572 | 19:57760018:G:A | ZNP805 | ZNF805_0.0 | 0.016984 | 11 |
| 8 | rs3746231 | 19:57760057:G:A | | | | |
| 9 | rs10421632 | 19:57764770:G:A | | | | |
| 10 | rs8100154 | 19:57765432:A:G | | | | |
| 11 | rs10045774 | 5:102891695:A:G | NUDT12 | NUDT12_0.0 | 0.016770 | 12 |
| 12 | rs4811 | 14:90730071:C:T | PSMC1 | PSMC1_0.0 | 0.015656 | 13 |
| 13 | rs687 | 2:68415767:G:A | PPP3R1 | PPP3R1_0.0 | 0.015592 | 14 |
| 14 | rs3829738 | 1:909309:T:C | PLEKHN1 | PLEKHN1_4.0 | 0.014456 | 15 |
| 15 | rs17099370 | 14:62463129:G:T | SYT16 | SYT16_2.0 | 0.012112 | 16 |
| 16 | rs2032360 | 12:52699548:T:C | KRT86 | KRT86_1.0 | 0.012036 | 17 |
| 17 | rs56204700 | 7:97821855:T:C | LMTK2 | LMTK2_1.0 | 0.012001 | 18 |
| 18 | rs3801296 | 7:97823125:G:A | | | | |
| 19 | rs10268268 | 7:97823629:A:G | | | | |
| 20 | rs6115 | 14:95053890:G:A | SERPINA5 | SERPINA5_3.0 | 0.011956 | 19 |
| 21 | rs6112 | 14:95054176:T:C | | | | |
| 22 | rs6116 | 14:95058462:A:C | | | | |
| 23 | rs5931046 | X:136112707:A:G | GPR101 | GPR101_2.0 | 0.011905 | 20 |
| 24 | rs2269415 | X:152823728:G:C | ATP2B3 | ATP2B3_1.0 | 0.011570 | 21 |
| 25 | rs10455097 | 6:74493432:A:C | CD109 | CD109_3.0 | 0.011321 | 22 |
| 26 | rs2917862 | 6:74521947:C:T | | | | |
| 27 | rs2694657 | 12:80190650:C:G | PPP1R12A | PPP1R12A_0.0 | 0.011247 | 23 |
| 28 | rs2232108 | 7:43916727:T:G | URGCP | URGCP_0.0 | 0.011087 | 24 |
| 29 | rs2232105 | 7:43917013:G:A | | | | |
| 30 | rs2232103 | 7:43917274:A:G | | | | |
| 31 | rs2291289 | 11:72408657:C:T | ARAP1 | ARAP1_4.0 | 0.010782 | 25 |
| 32 | rs17855750 | 16:28515228:A:C | IL27 | IL27_0.0 | 0.010656 | 26 |
| 33 | rs619483 | 6:4087934:G:C | C6orf201 | C6orf201_0.0 | 0.010562 | 27 |
| 34 | rs643232 | 6:4122249:C:A | | | | |
| 35 | rs1050457 | 19:731144:A:G | PALM | PALM_1.0 | 0.010433 | 28 |
| 36 | rs2238567 | 19:746409:G:A | | | | |
| 37 | rs3737002 | 1:207760773:C:T | CR1 | CR1_1.0 | 0.010343 | 29 |
| 38 | rs3737002 | 1:207760773:C:T | | | | |
| 39 | rs941909 | 10:102988345:G:A | LBX1 | LBX1_0.0 | 0.010231 | 30 |
| 40 | rs1054124 | 5:135388663:A:G | TGFBI | TGFBI_3.0 | 0.010217 | 31 |
| 41 | rs1133170 | 5:135391374:C:T | | | | |
| 42 | rs4669 | 5:135392426:T:C | | | | |
| 43 | rs4934 | 14:95080803:G:A | SERPINA3 | SERPINA3_0.0 | 0.009884 | 32 |
| 44 | rs1059369 | 19:18497141:T:A | GDF15 | GDF15_1.0 | 0.009828 | 33 |
| 45 | rs1804826 | 19:18499238:G:T | | | | |
| 46 | rs3751884 | 16:1524850:A:G | CLCN7 | CLCN7_1.0 | 0.009754 | 34 |
| 47 | rs289318 | 19:23159486:T:C | ZNF728 | ZNF728_0.0 | 0.009346 | 35 |
| 48 | rs2572023 | 7:99474427:A:G | OR2AE1 | OR2AE1_0.0 | 0.009341 | 36 |
| 49 | rs216250 | 12:311949:A:G | SLC6A12 | SLC6A12_0.0 | 0.009332 | 37 |
| 50 | rs526690 | 12:319111:T:C | | | | |
| 51 | rs557881 | 12:319125:A:G | | | | |
| 52 | rs10277 | 5:179264731:T:C | MRNIP | MRNIP_1.0 | 0.009253 | 38 |
| 53 | rs1650893 | 5:179280379:T:C | | | | |
| 54 | rs10060182 | 5:179285752:G:A | | | | |
| 55 | rs3745403 | 19:51871195:G:A | CLDND2 | CLDND2_0.0 | 0.009002 | 39 |
| 56 | rs2474328 | 10:134161633:A:G | LRRC27 | LRRC27_0.0 | 0.008993 | 40 |
| 57 | rs1042780 | 11:86161388:C:G | ME3 | ME3_0.0 | 0.008863 | 41 |
| 58 | rs10909567 | 2:130925088:C:T | SMPD4 | SMPD4_1.0 | 0.008594 | 42 |
| 59 | rs7252988 | 19:48800338:A:G | CCDC114 | CCDC114_3.0 | 0.008497 | 43 |
| 60 | rs343376 | 1:42693597:A:G | FOXJ3 | FOXJ3_0.0 | 0.008473 | 44 |
| 61 | rs12918876 | 16:88496008:T:C | ZNF469 | ZNF469_6.0 | 0.008424 | 45 |
| 62 | rs3812953 | 16:88502482:C:T | | | | |
| 63 | rs1134074 | 16:70395387:C:T | DDX19A | DDX19A_0.0 | 0.008357 | 46 |
| 64 | rs7380824 | 5:138856982:C:T | STING1 | STING1_1.0 | 0.008353 | 47 |
| 65 | rs78233829 | 5:138857925:C:G | | | | |
| 66 | rs11554776 | 5:138861078:C:T | | | | |
| 67 | rs1043836 | 9:116136198:C:T | HDHD3 | HDHD3_1.0 | 0.008140 | 48 |
| 68 | rs3739287 | 8:124525483:C:T | FBXO32 | FBXO32_0.0 | 0.007880 | 49 |
| 69 | rs2974617 | 5:114462355:C:T | TRIM36 | TRIM36_1.0 | 0.007865 | 50 |
| 70 | rs7716270 | 5:114480364:C:T | | | | |
| 71 | rs9462088 | 6:35430686:G:A | FANCE | FANCE_0.0 | 0.007706 | 52 |
| 72 | rs3729740 | 5:38496637:C:T | LIFR | LIFR_0.0 | 0.007609 | 53 |
| 73 | rs4906422 | 14:104641612:T:C | KIF26A | KIF26A_1.0 | 0.007528 | 54 |
| 74 | rs3742947 | 14:104642422:A:G | | | | |
| 75 | rs11621644 | 14:104644147:C:T | | | | |
| 76 | rs314452 | 9:114090279:A:G | OR2K2 | OR2K2_0.0 | 0.007415 | 55 |
| 77 | rs8102923 | 19:18375608:G:A | IQCN | IQCN_1.0 | 0.007297 | 56 |
| 78 | rs8103177 | 19:18375815:G:A | | | | |
| 79 | rs2277921 | 19:18375846:G:A | | | | |
| 80 | rs12608777 | 19:18375882:G:C | | | | |
| 81 | rs61740752 | 19:18376517:C:T | | | | |
| 82 | rs12609001 | 19:18377761:A:G | | | | |
| 83 | rs2232762 | 15:65347405:G:A | RASL12 | RASL12_0.0 | 0.007274 | 57 |
| 84 | rs1866844 | 8:95531419:T:C | VIRMA | VIRMA_0.0 | 0.007251 | 58 |
| 85 | rs2890827 | 8:95538468:T:C | | | | |
| 86 | rs957448 | 8:95541302:A:G | | | | |
| 87 | rs2304764 | 8:95547119:T:G | | | | |
| 88 | rs4085749 | 1:196920148:C:T | CFHR2 | CFHR2_0.0 | 0.007101 | 59 |
| 89 | rs1061770 | 1:32096265:A:G | PEF1 | PEF1_0.0 | 0.006982 | 60 |
| 90 | rs17857295 | 20:3838441:C:G | MAVS | MAVS_2.0 | 0.006971 | 61 |
| 91 | rs2326369 | 20:3842984:C:T | | | | |
| 92 | rs6779903 | 3:135720851:G:T | PPP2R3A | PPP2R3A_2.0 | 0.006821 | 62 |
| 93 | rs16843645 | 3:135745911:G:A | | | | |
| 94 | rs12943620 | 17:78444658:T:G | NPTX1 | NPTX1_2.0 | 0.006601 | 63 |
| 95 | rs1131609 | 17:78445702:A:G | | | | |
| 96 | rs2292954 | 16:89613123:A:G | SPG7 | SPG7_1.0 | 0.006584 | 64 |
| 97 | rs12960 | 16:89620328:G:A | | | | |
| 98 | rs8105737 | 19:15198631:A:C | OR1I1 | OR1I1_1.0 | 0.006398 | 65 |
| 99 | rs41291993 | 12:57039080:C:T | ATP5F1B | ATP5F1B_0.0 | 0.006236 | 66 |
| 100 | rs1132358 | 16:1397815:C:T | BAIAP3 | BAIAP3_0.0 | 0.006192 | 67 |
| 101 | rs231591 | 19:36224705:A:G | KMT2B | KMT2B_3.0 | 0.005946 | 68 |
| 102 | rs584961 | 11:75277628:A:G | SERPINH1 | SERPINH1_3.0 | 0.005552 | 69 |
| 103 | rs1133763 | 17:32647831:A:C | CCL8 | CCL8_1.0 | 0.005422 | 70 |
| 104 | rs2229276 | 1:237054569:A:G | MTR | MTR_4.0 | 0.005307 | 71 |
| 105 | rs12070777 | 1:237058744:C:A | | | | |
| 106 | rs5756130 | 22:36684331:C:T | MYH9 | MYH9_1.0 | 0.005296 | 72 |
| 107 | rs2269529 | 22:36684354:T:C | | | | |
| 108 | rs2269530 | 22:36684358:C:A | | | | |
| 109 | rs602128 | 11:102713465:A:G | MMP3 | MMP3_0.0 | 0.005277 | 73 |
| 110 | rs679620 | 11:102713620:T:C | | | | |
| 111 | rs63750222 | 17:44061025:C:T | MAPT | MAPT_2.0 | 0.004820 | 74 |
| 112 | rs61744212 | 15:22840279:G:T | TUBGCP5 | TUBGCP5_0.0 | 0.004813 | 75 |
| 113 | rs2306857 | 3:112727184:A:T | NEPRO | NEPRO_2.0 | 0.004733 | 76 |
| 114 | rs1979572 | 17:28511978:G:A | NSRP1 | NSRP1_1.0 | 0.004684 | 77 |
| 115 | rs2277923 | 5:172662024:T:C | NKX2-5 | NKX2-5_0.0 | 0.004416 | 78 |
| 116 | rs10867826 | 9:84607758:T:A | SPATA31D1 | SPATA31D1_1.0 | 0.004400 | 79 |
| 117 | rs2304155 | 19:11326119:A:G | DOCK6 | DOCK6_9.0 | 0.004355 | 80 |
| 118 | rs2304154 | 19:11326125:C:T | | | | |
| 119 | rs4804151 | 19:11327608:T:C | | | | |
| 120 | rs4804152 | 19:11327626:A:G | | | | |
| 121 | rs17847215 | 11:71941033:G:C | INPPL1 | INPPL1_1.0 | 0.004254 | 81 |
| 122 | rs3008815 | 6:39864730:C:T | DAAM2 | DAAM2_0.0 | 0.004248 | 82 |
| 123 | rs10821135 | 9:96238578:C:T | FAM120A | FAM120A_1.0 | 0.004118 | 83 |
| 124 | rs730106 | X:153221657:T:C | HCFC1 | HCFC1_1.0 | 0.004032 | 84 |
| 125 | rs5498 | 19:10395683:A:G | ICAM1 | ICAM1_1.0 | 0.004031 | 85 |
| 126 | rs639225 | 9:27202870:A:G | TEK | TEK_3.0 | 0.003944 | 86 |
| 127 | rs13058434 | 22:26164413:C:T | MYO18B | MYO18B_1.0 | 0.003813 | 87 |
| 128 | rs10753668 | 1:165218792:G:A | LMX1A | LMX1A_1.0 | 0.003744 | 88 |
| 129 | rs9449444 | 6:82900811:G:A | IBTK | IBTK_0.0 | 0.003400 | 89 |
| 130 | rs9361904 | 6:82933309:C:T | | | | |
| 131 | rs2273165 | 1:94341267:A:G | DNTTIP2 | DNTTIP2_3.0 | 0.003388 | 90 |
| 132 | rs231228 | 19:36268771:C:T | ARHGAP33 | ARHGAP33_4.0 | 0.003359 | 91 |
| 133 | rs35297478 | 19:36273308:G:A | | | | |
| 134 | rs7852399 | 9:34371788:A:T | MYORG | MYORG_2.0 | 0.003326 | 92 |
| 135 | rs8565 | 7:75630274:T:C | STYXL1 | STYXL1_2.0 | 0.002990 | 93 |
| 136 | rs3820285 | 1:22336277:C:G | CELA3A | CELA3A_0.0 | 0.002903 | 94 |
| 137 | rs2272251 | 7:143042837:C:T | CLCN1 | CLCN1_3.0 | 0.002734 | 95 |
| 138 | rs3746906 | 21:43327856:A:G | C2CD2 | C2CD2_1.0 | 0.002720 | 96 |
| 139 | rs231235 | 19:36278470:C:G | ARHGAP33 | ARHGAP33_5.0 | 0.002705 | 97 |
| 140 | rs1126605 | 12:7242204:C:T | C1R | C1R_3.0 | 0.002689 | 98 |
| 141 | rs3765114 | 3:113655207:C:T | GRAMD1C | GRAMD1C_1.0 | 0.002368 | 99 |
| 142 | rs8042919 | 15:50878630:G:A | TRPM7 | TRPM7_0.0 | 0.001083 | 100 |

| | | | | ESE/ | | | | | |
|---|---|---|---|---|---|---|---|---|---|
| | | | | ESS | Coding | | | | |
| | Expression | TF | miRNA | or | SNP | AA | | Haplotype | AA |
| No. | change | binding | binding | ISRE | type | Effect | CodonUseDiff | Size | Change |
| 1 | Y | | | E | S | | | 1 | T > T |
| 2 | Y | | | E | S | | | 1 | K > K |
| 3 | Y | | | E | N | B | | 1 | A > V |
| 4 | Y | | | | S | | | 1 | H > H |
| 5 | Y | | | E | N | B | | 1 | S > N |
| 6 | Y | | ? | E | S | | | 1 | N > N |
| 7 | Y | | | E | N | B | | 4 | G > E |
| 8 | Y | | | E | N | B | | | G > D |
| 9 | Y | | | E | N | B | | | V > I |
| 10 | Y | | | E | S | | | | K > K |
| 11 | Y | | | E | S | | | 1 | L > L |
| 12 | Y | ? | | E | S | | | 1 | I > I |
| 13 | Y | | | | S | | | 1 | D > D |
| 14 | Y | | | E | N | B | | 1 | S > P |
| 15 | | | | | N | B | | 1 | R > L |
| 16 | Y | | | E | S | | | 1 | A > A |
| 17 | Y | | | | N | B | | 3 | I > T |
| 18 | Y | ? | | E | S | | | | R > R |
| 19 | Y | | | E | S | | | | A > A |
| 20 | Y | | | E | N | B | | 3 | S > N |
| 21 | Y | ? | | E | S | | | | P > P |
| 22 | Y | | | E | S | | | | I > I |
| 23 | | | | | N | P | | 1 | L > P |
| 24 | | | | E | S | | | 1 | V > V |
| 25 | Y | | | E | N | B | | 2 | Y > S |
| 26 | Y | | | | N | B | | | T > M |
| 27 | Y | | | E | S | | | 1 | L > L |
| 28 | Y | | | | N | B | | 3 | M > L |
| 29 | Y | | | | S | | | | H > H |
| 30 | Y | | | | S | | | | H > H |
| 31 | Y | | | E | S | | | 1 | L > L |
| 32 | Y | +/− | | E | N | P | | 1 | S > A |
| 33 | Y | | | E | N | B | | 2 | R > P |
| 34 | Y | | | E | N | B | | | N > K |
| 35 | Y | | | E | N | B | | 2 | T > A |
| 36 | | | | E | S | | | | A > A |
| 37 | Y | | | | N | P | | 2 | T > M |
| 38 | Y | | | | N | P | | | T > M |
| 39 | Y | ? | | | S | | | 1 | R > R |
| 40 | Y | | | E | S | | + | 3 | V > V |
| 41 | Y | | | | S | | | | L > L |
| 42 | Y | ? | | | S | | | | F > F |
| 43 | Y | | | | N | B | | 1 | A > T |
| 44 | Y | | | E | N | P | | 2 | S > T |
| 45 | Y | ? | | E | S | | | | P > P |
| 46 | Y | | | | S | | | 1 | P > P |
| 47 | Y | | | E | N | P | | 1 | Y > C |
| 48 | Y | | | E | N | B | | 1 | I > T |
| 49 | Y | | | E | S | | | 3 | T > T |
| 50 | Y | | | | S | | | | A > A |
| 51 | Y | | | | N | B | | | C > R |
| 52 | Y | | | | N | B | | 3 | Q > R |
| 53 | Y | | | | N | B | | | Q > R |
| 54 | Y | +/− | | E | S | | | | C > C |
| 55 | Y | +/− | | E | S | | | 1 | C > C |
| 56 | Y | | | | S | | | 1 | P > P |
| 57 | Y | | | E | N | B | | 1 | K > N |
| 58 | Y | | | E | S | | | 1 | T > T |
| 59 | Y | | | | S | | | 1 | S > S |
| 60 | Y | | | E | N | B | | 1 | V > A |
| 61 | Y | | | | S | | | 2 | P > P |
| 62 | | | | | S | | | | R > R |
| 63 | Y | | | E | S | | | 1 | N > N |
| 64 | Y | | | | N | D | | 3 | R > Q |
| 65 | Y | | | | N | B | | | G > A |
| 66 | Y | | | | N | B | | | R > H |
| 67 | Y | − | | E | N | B | | 1 | G > E |
| 68 | Y | | | E | S | | | 1 | T > T |
| 69 | Y | + | | | N | B | | 2 | D > N |
| 70 | Y | +/− | | | S | | − | | L > L |
| 71 | Y | | | E | N | B | | 1 | A > T |
| 72 | | | | E | N | B | | 1 | D > N |
| 73 | Y | | | | S | | | 3 | G > G |
| 74 | Y | | | | S | | | | A > A |
| 75 | | | | | S | | | | S > S |
| 76 | Y | ? | | | S | | | 1 | A > A |
| 77 | Y | ? | | | S | | | 6 | A > A |
| 78 | Y | | | E | S | | | | N > N |
| 79 | Y | | | | N | B | | | P > L |
| 80 | Y | | | | N | D | | | P > R |
| 81 | Y | | | | S | | | | Q > Q |
| 82 | Y | | | | N | B | | | C > R |
| 83 | Y | − | | | S | | | 1 | T > T |
| 84 | Y | | | | S | | | 4 | Q > Q |
| 85 | Y | ? | | E | S | | | | P > P |
| 86 | Y | ? | | E | S | | | | G > G |
| 87 | Y | | | | S | | | | P > P |
| 88 | Y | | | E | S | | | 1 | C > C |
| 89 | Y | + | | E | S | | | 1 | I > I |
| 90 | Y | | | | N | P | | 2 | Q > E |
| 91 | Y | | | E | S | | | | D > D |
| 92 | Y | | | | N | B | | 2 | A > S |
| 93 | | | | E | N | B | | | D > N |
| 94 | Y | | | | S | | | 2 | G > G |
| 95 | Y | ? | | | S | | | | L > L |
| 96 | Y | | | | N | B | | 2 | T > A |
| 97 | Y | − | | | N | B | | | R > Q |
| 98 | Y | ? | | | N | D | | 1 | Y > S |
| 99 | | | | | N | D | | 1 | R > H |
| 100 | Y | | | | S | | | 1 | D > D |
| 101 | Y | | | E | N | B | | 1 | D > G |
| 102 | Y | | | | S | | + | 1 | L > L |
| 103 | Y | | | E | N | P | | 1 | K > Q |
| 104 | Y | | | | S | | | 2 | A > A |
| 105 | Y | | | | S | | | | R > R |
| 106 | Y | | | E | S | | | 3 | R > R |
| 107 | Y | | | E | N | B | | | I > V |
| 108 | Y | | | | S | | | | A > A |
| 109 | Y | | | E | S | | | 2 | D > D |
| 110 | Y | | | E | N | B | | | K > E |
| 111 | Y | | | | S | | | 1 | D > D |
| 112 | Y | | | | S | | | 1 | L > L |
| 113 | Y | | | | N | B | | 1 | F > I |
| 114 | Y | | | E | S | | | 1 | Q > Q |
| 115 | Y | | | E | S | | | 1 | E > E |
| 116 | Y | | | E | S | | | 1 | T > T |
| 117 | Y | ? | | E | S | | | 4 | N > N |
| 118 | Y | | | E | S | | | | P > P |
| 119 | Y | | | E | S | | + | | L > L |
| 120 | Y | | | E | S | | | | D > D |
| 121 | | | | | N | B | | 1 | K > N |
| 122 | Y | | | E | S | | | 1 | I > I |
| 123 | Y | | | E | S | | | 1 | H > H |
| 124 | | ? | | E | S | | | 1 | P > P |
| 125 | Y | | | | N | B | | 1 | K > E |
| 126 | Y | | | E | S | | | 1 | S > S |
| 127 | Y | | | | N | B | | 1 | P > L |
| 128 | | ? | | E | S | | − | 1 | L > L |
| 129 | Y | | ? | E | N | B | | 2 | A > V |
| 130 | Y | | | E | S | | | | K > K |
| 131 | Y | | ? | E | S | | | 1 | F > F |
| 132 | Y | | | | S | | | 2 | A > A |
| 133 | Y | | | E | S | | | | L > L |
| 134 | Y | ? | | | N | D | | 1 | F > Y |
| 135 | Y | | | | S | | + | 1 | Q > Q |
| 136 | Y | | | | N | B | | 1 | A > G |
| 137 | Y | | | E | S | | | 1 | D > D |
| 138 | Y | | | E | S | | | 1 | F > F |
| 139 | Y | | | | S | | | 1 | A > A |
| 140 | Y | | | | N | B | | 1 | E > K |
| 141 | Y | ? | | | S | | + | 1 | N > N |
| 142 | Y | | | | N | B | | 1 | T > I |

Non-Genetic Features
Platelet Count (K)
Haemoglobin Value (g/dL)
Duration of Morning Stiffness (mins)
Age (Years)
Anti-cyclic Citrullinated Peptides (anti-CCP)
Legend:
Y Yes
N Nonsynonymous SNV
S Synonymous SNV
+ Create TF/miRNA binding site
− Delete TF/miRNA binding site
Unknown effect
E ESE/ESS
I ISRE
B Benign effect on AA
P Possibly damaging effect on AA
D Deleterious effect on AA

TABLE 3
Functional Characteristics of 9 SNPs that are predictive of rheumatoid arthritis

| | | | | | Mean | | Univariate Logistic Regression | | | | |
|---|---|---|---|---|---|---|---|---|---|---|---|
| | | | | | Feature | Feature | | | β | | |
| | | | | | Importance | Importance | Odds | 95% | (Effect | | Localised |
| # | SNP rsID | Chromosome | Position | Gene | Score | Ranking | Ratio | CI | size) | P-value | Region |
| 1 | rs1266832853 | 1 | 248722788 | OR2T29; | 0.0612 | 1 | 33.19 | (23.7, | 3.502 | 2.57E−92 | Exon |
| | | | | OR2T5 | | | | 46.49) | | | |
| 2 | rs200901373 | 2 | 130872956 | POTEF | 0.0501 | 2 | 81.49 | (47.68, | 4.400 | 3.07E−58 | Intron |
| | | | | | | | | 139.3) | | | |
| 3 | rs60465633 | 1 | 16918473 | NBPF1 | 0.0486 | 3 | 1448 | (202.4, | 7.278 | 4.20E−13 | Exon |
| | | | | | | | | 10360) | | | |
| 4 | rs142869031 | 15 | 43906035 | STRC | 0.0456 | 4 | 0.0081 | (0.0030, | −4.822 | 1.02E−21 | Intron |
| | | | | | | | | 0.0216) | | | |
| 5 | rs11385557 | 7 | 97944934 | BAIAP2L1 | 0.0365 | 5 | 0.0137 | (0.0061, | −4.290 | 2.49E−25 | Intron |
| | | | | | | | | 0.0308) | | | |
| 6 | rs143773270 | 10 | 18085101 | TMEM236 | 0.0328 | 6 | 0.0053 | (0.0013, | −5.238 | 1.56E−13 | Exon |
| | | | | | | | | 0.0213) | | | |
| 7 | rs11246240 | 11 | 651491 | DEAF1 | 0.0268 | 7 | 0.1083 | (0.0780, | −2.223 | 5.51E−40 | Intron |
| | | | | | | | | 0.1506) | | | |
| 8 | rs112667995 | 19 | 14675207 | TECR | 0.0253 | 8 | 0.0503 | (0.0308, | −2.990 | 4.52E−33 | Intron |
| | | | | | | | | 0.0820) | | | |
| 9 | rs79555231 | 6 | 162992348 | PRKN | 0.0238 | 9 | 0.0825 | (0.0567, | −2.495 | 7.55E−39 | Intron |
| | | | | | | | | 0.1200) | | | |

| | Predicted | pfSNP/ | | | | | | | | |
|---|---|---|---|---|---|---|---|---|---|---|
| | Function | LD | | | Gene | | | Endocrine | Cellular | Pathways |
| # | (pfSNP)? | Details | Metabolism | Immunity | Expression | Pharmacology | Disease | System | Function | (Concised) |
| 1 | ✓ | Non- | | | | | | | | Signal |
| | | Synonymous | | | | | | | | transduction |
| | | A > D | | | | | | | | Olfactory |
| | | Tolerated - Low | | | | | | | | transduction |
| | | Confidence | | | | | | | | |
| 2 | | | | | | | | | | |
| 3 | ✓ | Non- | | | | | | | | |
| | | Synonymous | | | | | | | | |
| | | T > M | | | | | | | | |
| | | Tolerated - Low | | | | | | | | |
| | | Confidence | | | | | | | | |
| 4 | LD | 5 pfSNPs in LD | | | | | | | | Sensory Perception |
| 5 | LD | 19 pfSNPs | ✓ | | ✓ | | ✓ | ✓ | | Signal transduction |
| | | in LD | | | | | | | | |
| 6 | ✓ | Non- | | | | | | | | |
| | | Synonymous | | | | | | | | |
| | | G > R | | | | | | | | |
| | | Deleterious | | | | | | | | |
| 7 | ✓ | eQTL | | | ✓ | | | | | SIDS |
| | | | | | | | | | | Susceptiblity |
| | | | | | | | | | | Pathways |
| 8 | ✓ | eQTL | ✓ | | ✓ | | | | ✓ | Metabolism |
| | | | | | | | | | | of Lipids |
| 9 | LD | 21 pfSNPs | | | | | | | | Metabolism |
| | | in LD | | | | | | | | of Proteins |
| | | | | | | | | | | Adaptive |
| | | | | | | | | | | Immune |
| | | | | | | | | | | System |
| | | | | | | | | | | Mitophagy |

TABLE 4
Functional Characteristics of additional 4 SNPs that are predictive of rheumatoid arthritis

| | | | | | Univariate Logistic | | | | |
|---|---|---|---|---|---|---|---|---|---|
| | | | Mean | | Regression | | | | |
| | | | Feature | Feature | β | | | Predicted | pfSNP/ |
| | | | Importance | Importance | (Effect | | Localised | Function | LD |
| # | SNP rsID | Gene | Score | Ranking | size) | P-value | Region | (pfSNP)? | Details |
| 1 | rs879004458 | CASC18; | 0.0201 | 10 | 2.944 | 8.44E−63 | Intergenic | | |
| | | NUAK1 | | | | | | | |
| 2 | rs68004247 | TRPM4 | 0.0192 | 11 | −2.365 | 5.25E−40 | Intron | ✓ | eQTL |
| 3 | rs151105471 | DCAF4 | 0.0140 | 12 | −1.742 | 7.56E−33 | Intron | ✓ | eQTL |
| 4 | 15:82890763:C:A | ADAMTS7P2 | 0.0118 | 13 | −2.646 | 2.22E−23 | Pseudo- | LD | 1 pfSNP |
| | | | | | | | gene | | in LD |

| | | | Gene | | | Endocrine | Cellular | Pathways |
|---|---|---|---|---|---|---|---|---|
| # | Metabolism | Immunity | Expression | Pharmacology | Disease | System | Function | (Concised) |
| 1 | | | | | | | | Gene Expression |
| | | | | | | | | (Transcription) |
| 2 | | | | | | | | Sensory |
| | | | | | | | | Perception |
| | | | | | | | | Transport |
| | | | | | | | | of small |
| | | | | | | | | molecules |
| 3 | ✓ | | ✓ | ✓ | | | ✓ | Post- |
| | | | | | | | | translational |
| | | | | | | | | protein |
| | | | | | | | | modifications |
| 4 | | | | | | | | |

TABLE 5
Predictive performance of the 9 selected SNPs in
a 5-fold cross-validation of the Train dataset

| | | Machine Learning Models | | | | |
|---|---|---|---|---|---|---|
| | | Logistic | Naïve | Random | | SVM |
| Dataset | Evaluation Metric | Regression | Bayes | Forest | XGBoost | RBF |
| Training set | Mean AUC | 0.992 | 0.990 | 0.994 | 0.994 | 0.992 |
| Cross- | Mean Sensitivity | 0.968 | 0.975 | 0.975 | 0.973 | 0.968 |
| validation | Mean Specificity | 0.963 | 0.956 | 0.962 | 0.963 | 0.965 |
| | Mean Accuracy | 0.966 | 0.966 | 0.968 | 0.968 | 0.966 |

TABLE 6
Predictive performance of the 9 selected SNPs
in each of the 3 unseen Test datasets

| | | Machine Learning Models | | | | |
|---|---|---|---|---|---|---|
| | Evaluation | Logistic | Naïve | Random | | SVM |
| Dataset | Metric | Regression | Bayes | Forest | XGBoost | RBF |
| Test1 | AUC | 0.990 | 0.986 | 0.993 | 0.994 | 0.991 |
| Evaluation | Sensitivity | 0.928 | 0.936 | 0.928 | 0.936 | 0.936 |
| | Specificity | 0.963 | 0.960 | 0.963 | 0.963 | 0.963 |
| | F1 Score | 0.913 | 0.914 | 0.913 | 0.918 | 0.918 |
| | Accuracy | 0.945 | 0.948 | 0.945 | 0.949 | 0.949 |
| | Avg. Precision | 0.970 | 0.951 | 0.975 | 0.976 | 0.970 |
| Test2 | AUC | 0.988 | 0.982 | 0.989 | 0.992 | 0.990 |
| Evaluation | Sensitivity | 0.937 | 0.945 | 0.945 | 0.945 | 0.937 |
| | Specificity | 0.961 | 0.961 | 0.963 | 0.963 | 0.963 |
| | F1 Score | 0.915 | 0.920 | 0.923 | 0.923 | 0.919 |
| | Accuracy | 0.949 | 0.953 | 0.954 | 0.954 | 0.950 |
| | Avg. Precision | 0.964 | 0.960 | 0.968 | 0.972 | 0.965 |
| Test3 | AUC | 0.987 | 0.972 | 0.987 | 0.991 | 0.986 |
| Evaluation | Sensitivity | 0.913 | 0.921 | 0.921 | 0.937 | 0.921 |
| | Specificity | 0.966 | 0.958 | 0.966 | 0.966 | 0.966 |
| | F1 Score | 0.910 | 0.903 | 0.914 | 0.922 | 0.914 |
| | Accuracy | 0.940 | 0.940 | 0.944 | 0.952 | 0.944 |
| | Avg. Precision | 0.961 | 0.936 | 0.958 | 0.965 | 0.946 |

TABLE 7
Predictive performance of Polygenic Risk Scores (PRS) calculated
from the 9 selected SNPs in a 5-fold cross-validation of the
Training dataset and in each of the 3 unseen Test datasets.

| | | Machine Learning Models | | | | |
|---|---|---|---|---|---|---|
| | Evaluation | Logistic | Naïve | Random | | SVM |
| Dataset | Metric | Regression | Bayes | Forest | XGBoost | RBF |
| Training set | Mean AUC | 0.992 | 0.992 | 0.992 | 0.992 | 0.982 |
| Cross- | Mean Sensitivity | 0.967 | 0.965 | 0.970 | 0.972 | 0.972 |
| validation | Mean Specificity | 0.964 | 0.964 | 0.961 | 0.960 | 0.962 |
| | Mean Accuracy | 0.965 | 0.965 | 0.966 | 0.966 | 0.967 |
| | Mean Average | 0.980 | 0.980 | 0.976 | 0.972 | 0.968 |
| | Precision | | | | | |
| | (PR-AUC) | | | | | |
| Test1 | AUC | 0.994 | 0.994 | 0.993 | 0.992 | 0.990 |
| Evaluation | Sensitivity | 0.992 | 0.992 | 0.976 | 0.976 | 0.992 |
| | Specificity | 0.963 | 0.963 | 0.954 | 0.957 | 0.960 |
| | F1 Score | 0.947 | 0.947 | 0.928 | 0.931 | 0.943 |
| | Accuracy | 0.977 | 0.977 | 0.965 | 0.967 | 0.976 |
| | Avg. Precision | 0.977 | 0.977 | 0.973 | 0.972 | 0.970 |
| Test2 | AUC | 0.988 | 0.988 | 0.986 | 0.989 | 0.978 |
| Evaluation | Sensitivity | 0.945 | 0.945 | 0.969 | 0.969 | 0.969 |
| | Specificity | 0.961 | 0.961 | 0.963 | 0.963 | 0.961 |
| | F1 Score | 0.920 | 0.920 | 0.935 | 0.935 | 0.932 |
| | Accuracy | 0.953 | 0.953 | 0.966 | 0.966 | 0.965 |
| | Avg. Precision | 0.962 | 0.962 | 0.962 | 0.966 | 0.955 |
| Test3 | AUC | 0.984 | 0.984 | 0.980 | 0.983 | 0.983 |
| Evaluation | Sensitivity | 0.961 | 0.961 | 0.976 | 0.976 | 0.969 |
| | Specificity | 0.966 | 0.966 | 0.963 | 0.966 | 0.966 |
| | F1 Score | 0.935 | 0.935 | 0.939 | 0.943 | 0.939 |
| | Accuracy | 0.963 | 0.963 | 0.970 | 0.971 | 0.967 |
| | Avg. Precision | 0.959 | 0.959 | 0.949 | 0.955 | 0.953 |

1. A method of predicting the responsiveness of a subject towards methotrexate (MTX) treatment, the method comprising a) detecting the allele and the number of alleles at a plurality of SNP loci listed in Table 1 or Table 2, using a sample obtained from the subject, and b) generating a risk score for the subject based on the allele and the number of alleles at the plurality of SNP loci, wherein the risk score, as compared to a reference, provides an indication as to whether the subject is responsive or non-responsive towards MTX treatment.

2. The method of claim 1, wherein the risk score is a polygenic risk score.

3. The method of any one of claim 1 or 2, wherein the risk score is calculated by weighting the allele and number of alleles at the plurality of SNP loci.

4. The method of any one of claims 1 to 3, wherein the risk score is calculated by an algorithm.

5. The method of any one of claims 1 to 4, wherein the method further comprises the use of five non-genetic factors to generate a risk score for the subject.

6. The method of any one of claims 1 to 5, wherein the subject is suffering from rheumatoid arthritis (RA).

7. The method of any one of claims 1 to 6, wherein the sample is a blood sample.

8. A method of treating a subject in need of therapy, the method comprising a) detecting the allele and the number of alleles at a plurality of SNP loci listed in Table 1 or Table 2, using a sample obtained from the subject; b) generating a risk score for the subject based on the allele and the number of alleles at the plurality of SNP loci, wherein the risk score, as compared to a reference, provides an indication as to whether the subject is responsive or non-responsive towards MTX treatment; and c) treating the subject when the subject is predicted to be responsive or non-responsive towards MTX.

9. A method of detecting a subject at risk of rheumatoid arthritis, the method comprising a) detecting the allele and the number of alleles at a plurality of SNP loci listed in Table 3, using a sample obtained from the subject, and b) generating a risk score for the subject based on the allele and the number of alleles at the plurality of SNP loci, wherein the risk score, as compared to a reference, provides an indication as to whether the subject is at risk of rheumatoid arthritis.

10. The method of claim 9, wherein the score is a polygeneic risk score.

11. The method of claim 9 or 10, further comprising detecting, in a sample obtained from the subject, the allele and the number of alleles at a plurality of SNP loci listed in Table 4 to generate a risk score for the subject.

12. A method of treating a subject at risk of rheumatoid arthritis, the method comprising a) detecting the allele and the number of alleles at a plurality of SNP loci listed in Table 3, using a sample obtained from the subject; b) generating a risk score for the subject based on the allele and the number of alleles at the plurality of SNP loci, wherein the risk score, as compared to a reference, provides an indication as to whether the subject is at risk of rheumatoid arthritis; and c) treating the subject found to be at risk of rheumatoid arthritis.

13. A computer-implemented method for determining a genetic signature for predicting the responsiveness of a subject towards methotrexate (MTX) treatment, the method comprising:
a) receiving exome sequencing records of a plurality of subjects responsive towards MTX treatment (case records) and a plurality of subjects not responsive to MTX treatment (control records), wherein the case records and control records are identified with case record and control record labels;
b) merging the case records and control records to obtain a training dataset;
c) aligning records in the training dataset to the human reference genome to obtain an aligned training dataset;
d) identifying potentially functional SNPs (pfSNPs) and/or coding haplotypes in each of the records in the aligned training dataset, wherein each identified pfSNP and/or coding haplotype is treated as a candidate feature;
e) performing feature selection among the pfSNPs and/or coding haplotypes using a recursive feature selection algorithm and the case and control record labels to select a plurality of predictive pfSNPs and/or coding haplotypes predictive of responsiveness towards MTX treatment;
f) defining a genetic signature based on the selected plurality of predictive pfSNPs and/or coding haplotypes predictive of responsiveness towards MTX treatment.

14. A computer-implemented method for determining a genetic signature for predicting a subject at risk of rheumatoid arthritis, the method comprising:
a) receiving exome sequencing records of a plurality of subjects suffering from rheumatoid arthritis (case records) and a plurality of subjects not suffering from rheumatoid arthritis (control records), wherein the case records and control records are identified with case record and control record labels;
b) merging the case records and control records to obtain a training dataset;
c) aligning records in the training dataset to the human reference genome to obtain an aligned training dataset;
d) identifying SNPs in each of the records in the aligned training dataset, wherein each identified SNP is treated as a candidate feature;
e) performing feature selection among the SNPs using a recursive feature selection algorithm and the case and control record labels to select a plurality of predictive SNPs predictive of rheumatoid arthritis;
f) defining a genetic signature based on the selected plurality of predictive SNPs predictive of rheumatoid arthritis.
